(19) United States
(12) Patent Application Publication
(10) Pub. No.: US 2003/0096281 A1
(43) Pub. Date: May 22, 2003

(54) Methods of making glycomolecules with enhanced activities and uses thereof

(76) Inventors: Ganesh Venkataraman, Bedford, MA (US); Ram Sasisekharan, Cambridge, MA (US); Zachary Shriver, Boston, MA (US)

Correspondence Address: FISH & RICHARDSON PC, 225 FRANKLIN ST, BOSTON, MA, 02110, US

(21) Appl. No.: 10/244,805

(22) Filed: Sep. 16, 2002

Related U.S. Application Data
(60) Provisional application No. 60/322,232, filed on Sep. 14, 2001.

(51) Int. Cl. C12Q 1/68; G06F 19/00; G01N 33/48; G01N 33/50; C07H 21/04; C12P 19/34; C12P 19/04; C08B 37/00

(52) U.S. Cl. 435/6; 435/91.2; 435/101; 536/23.1; 536/123; 702/20

(57) ABSTRACT

Methods to rapidly produce and identify polysaccharides, and other sugar structures, having enhanced activities, have been developed. The methods include producing a molecule, e.g., a therapeutic molecule, which includes a first, non-saccharide moiety (e.g., a protein, polypeptide, peptide, amino acid or lipid) and a second, polysaccharide, moiety. The method includes: determining the chemical composition and structure of all or a portion of the second moiety, modifying the structure of the second moiety to provide a modified second moiety, and evaluating or screening the molecule having the modified second moiety, e.g., for a biological activity or other chemical or physical property. In some embodiments, the step of determining the chemical structure and composition of the second moiety includes a comparison of one or more properties of the second moiety with a database, e.g., a database which correlates such one or more properties with structure or function of a polysaccharide.

This application claims priority to U.S. provisional application No. 60/322,232 filed on Sep. 14, 2001, the contents of which is incorporated herein by reference.

### BACKGROUND OF THE INVENTION

Glycomics is the study of sugars, information dense molecules that occur, in both linear and branched forms, in isolated form, as a structure on a cell or organelle, or on molecules such as proteins (referred to as glycoproteins) or lipids (referred to as glycolipids). Linear sugars are found on cell surfaces, attached to proteins and lipids and provide characteristic cellular signatures, mediate cell-cell communications, and actively orchestrate intracellular signal transduction. Branched sugars are found on protein surfaces, among other biopolymers, and provide characteristic protein signatures, mediate protein localization and targeting, and actively modulate protein efficacy, stability pharmacokinetics, and/or therapeutic (clinical) potency.

Although the importance of polysaccharides and other sugars has been recognized, the biotechnology field has not focused on these structures, largely due to the lack of technology enabling such a focus, and has not developed methods for automated sequencing, synthesis, or screening for biological activities. Instead, work has been performed on an individual basis, where a target is identified, analyzed as a polysaccharide or as having an important sugar component, the sugar composition and structure determined, and then analyzed for activity. Few attempts to enhance activity have been made, and typically only by changing a first sugar, then a second sugar, etc. This has been done chemically, by synthesizing molecules with different sugar compositions, or by using one or more different biological systems, for example, by altering or providing one or more enzymes involved in the synthesis of the polysaccharide, or by altering substrate availability.

### SUMMARY OF THE INVENTION

Methods to rapidly produce and identify polysaccharides, and other sugar structures, associated with glycomolecules having enhanced activities, have been developed. The methods include the steps of determining the chemical composition and structure of a polysaccharide moiety, e.g., a polysaccharide moiety having a defined activity, to analyze the sequence of sugars on molecules such as proteins, polypeptides and lipids, modifying the chemical composition or structure of the polysaccharide moiety, using for example enzymatic or solid-phase methods, and screening the modified polysaccharide moiety as part of a glycomolecule, for altered activity of the glycomolecule. Preferably, multiple features including structure, composition, and reactivity of the polysaccharide moiety is determined. The information obtained can then be used to synthesize polysaccharide moieties of interest using, e.g., enzymatic, chemical, or chemoenzymatic synthesis. In addition, the structure or composition can optionally be modified, and then re-screened for altered activity of a glycomolecule such as a glycoprotein, proteoglycan, glycopeptide or glycolipid.

Accordingly, in one aspect, the invention features a method for producing a molecule, e.g., a therapeutic molecule, which includes at least a first, non-saccharide moiety (e.g., a protein, polypeptide, peptide, amino acid or lipid) and a second, polysaccharide, moiety. The method includes: determining the chemical composition and structure of all or a portion of the second moiety, modifying the structure of the second moiety to provide a modified second moiety, and evaluating or screening the molecule having the modified second moiety, e.g., for a biological activity or other chemical or physical property. In some embodiments, the step of determining the chemical structure and composition of the second moiety includes a comparison of one or more properties of the second moiety with a database, e.g., a database which correlates such one or more properties with structure or function of a polysaccharide.

In some embodiments, the second polysaccharide moiety has a defined activity, e.g., an activity defined by comparison to a database of known polysaccharides, and the evaluation or screening includes evaluating the molecule for altered, enhanced or optimized biological activity of the modified second moiety.

In some embodiments, the chemical structure and composition of the second moiety is determined by comparing the length and/or molecular mass of the second moiety to a database of polysaccharides having known length and/or molecular mass; selecting from the database a subpopulation of polysaccharides having the same length or a similar molecular mass as the second moiety; applying an experimental constraint to the second moiety to determine a property of the second moiety; comparing the property of the second moiety to the subpopulation; and eliminating from the subpopulation polysaccharides which do not have the property of the second moiety when subjected to the same experimental constraint. This process can be repeated one or more times using a different experimental constraint and to thereby eliminate additional polysaccharides from the subpopulation.

Experimental constraints can include: enzymatic digestion, e.g., with an exoenzyme, an endoenzyme, chemical digestion, chemical modification, chemical peeling, interaction with a binding compound, and enzymatic modification, e.g., sulfonation at a particular position. Examples of enzymes which can be used to digest the polysaccharide moiety include α-galactosidase to cleave α1→3 glycosidic linkage after a galactose, β-galactosidase to cleave a β1→4 linkage after a galactose, an α2→3 sialidase to cleave a α2→3 glycosidic linkage after a sialic acid, an α2→6 sialidase to cleave after an α2→6 linkage after a sialic acid, an α1→2 fucosidase to cleave a α1→2 glycosidic linkage after a fucose, a α1→3 fucosidase to cleave a α1→3 glycosidic linkage after a fucose, an α1→4 fucosidase to cleave a α1→4 glycosidic linkage after a fucose, an α1→6 fucosidase to cleave an α1→6 glycosidic linkage after a fucose, β-N-Acetylhexosaminidase to cleave non-reducing terminal β1→2,3,4,6 linked N-acetylglucosamine, and N-acetylgalactosamine, alpha-N-Acetylgalactosaminidase to cleave terminal alpha 1→3 linked N-acetylgalactosamine from glycoproteins. Other enzymes such as aspartyl-N-acetylglucosaminidase can be used to cleave at a beta linkage after a GlcNAc in the core sequence of N-linked oligosaccharides.

Properties of the saccharide which can be determined include: the mass of part or all of the oligosaccharide structure, the charges of the chemical units of the saccharide, identities of the chemical units of the saccharide, confirmations of the chemical units of the saccharide, total charge of the saccharide, total number of sulfates of the saccharide, total number of acetates, total number of phosphates, presence and number of carboxylates, presence and number of aldehydes or ketones, dye-binding of the saccharide, compositional ratios of substitutents of the saccharide, compositional ratios of anionic to neutral sugars, presence of uronic acid, enzymatic sensitivity, linkages between chemical units of the saccharide, charge, branch points, number of branches, number of chemical units in each branch, core structure of a branched or unbranched saccharide, the hydrophobicity and/or charge/charge density of each branch, absence or presence of GlcNAc and/or fucose in the core of a branched saccharide, number of mannose in an extended core of a branched saccharide, presence or absence or sialic acid on a branched chain of a saccharide, the presence or absence of galactose on a branched chain of a saccharide.

In some embodiments, the method includes using the determined composition and structure of the second moiety to produce the modified second moiety or a portion thereof using enzymatic, chemical, or chemoenzymatic synthesis, or any combination thereof. In other embodiments, the modification of the second moiety includes using the determined composition and structure of the second moiety to produce the modified second moiety or portion thereof using metabolic engineering or any combination of the above.

The modification of the second moiety can include, e.g., changing one or more of the identity, number, or linkage of one or more chemical units in the second moiety. For instance, in some embodiments, the modification includes changing the number of branches in the second moiety. The polysaccharide moiety can be modified, e.g., by removing one or more branches from a polysaccharide (e.g., an endoglycan such as EndoF2 can be used to remove a branch from a biantennary polysaccharide) or adding one or more branches to a polysaccharide moiety (e.g., a core α1→6 fucose or β1→4 GlcNAc can be added to a polysaccharide moiety). Additional monosaccharides can be added to the additional branch or branches of the modified polysaccharide moiety.

In another embodiment, the polysaccharide moiety is enzymatically modified, e.g., by enzymatic cleavage and/or enzymatic addition of one or more chemical units.

In one embodiment, a polysaccharide moiety can be modified by enzymatically removing one or more chemical unit(s) of the polysaccharide, e.g., one or more of a sialic acid, fucose, galactose, glucose, xylose, GlcNAc, and/or a GalNAc can be removed from the polysaccharide moiety. Examples of enzymes which can be used to remove a chemical unit from the polysaccharide moiety include: α-galactosidase to cleave a α1→3 glycosidic linkage after a galactose, β-galactosidase to cleave aβ1→4 linkage after a galactose, an α2→3 sialidase to cleave a α2→3 glycosidic linkage after a sialic acid, an α2→6 sialidase to cleave after an α2→6 linkage after a sialic acid, an α1→2 fucosidase to cleave a α1→2 glycosidic linkage after a fucose, a α1→3 fucosidase to cleave a α1→3 glycosidic linkage after a fucose, an α1→4 fucosidase to cleave a α1→4 glycosidic linkage after a fucose, an α1→6 fucosidase to cleave an α1→6 glycosidic linkage after a fucose, a N-acetylglucosiaminidase to cleave a β1→2, a β1→4 or β1→6 linkage after a GlcNAc.

In another embodiment, a polysaccharide moiety can be modified by enzymatically adding one or more chemical unit(s) to the polysaccharide, e.g., one or more of a sialic acid, fucose, galactose, glucose, xylose, GlcNAc, and/or a GalNAc can be added to the polysaccharide moiety. Examples of enzymes which can be used to add a chemical unit include: sialyltransferase, e.g., α2→3 sialyltransferase or α2→6 sialyltransferase, fucosyltransferase, e.g., α1→2 fucosyltransferse, α1→3 fucosyltransferase, α1→4 fucosyltransferase or α1→6 fucosyltransferase, galactosyltransferase (e.g., α1→3 galactosyltransferase, β1→4 galactosyltransferase or β1→3 galactosyltransferase) and a N-acetylglucosaminyltransferase (e.g., N-acetylglucosaminyltransferase I, II or III).

In other embodiments, a polysaccharide moiety can be modified by removing one or more chemical units and adding one or more chemical units to the polysaccharide moiety. In another embodiment, the polysaccharide can be modified by altering one or more substituent associated with the polysaccharide, e.g., a chemical unit of a polysaccharide. For example, sulfonation, e.g., of a sialic acid, can be modified to add a sulfate, e.g., using a sulfatransferase, or by removing a sulfate, e.g., a sulfatase.

In another embodiment, the modification of the polysaccharide moiety can be effected by altering a synthetic process which produces a polysaccharide moiety, e.g., by adding an excess of a substrate or intermediate in a synthetic reaction. For example, one or more of a sialic acid, fucose, galactose, glucose, xylose, GlcNAc, and/or a GalNAc can be added to the polysaccharide moiety by adding one or more of these monosaccharides, e.g., activated forms of these monosaccharides or precursors to these monosaccharides, to a cell, e.g., a recombinant cell which produces the polysaccharide to be modified. In addition, an enzyme which incorporates a chemical unit into a polysaccharide chain can be added. Examples of enzymes which can be used to add a chemical unit include: sialyltransferase, e.g., α2→3 sialyltransferase or α2→6 sialyltransferase, fucosyltransferase, e.g., α1→2 fucosyltransferse, α1→3 fucosyltransferase, α1→4 fucosyltransferase or α1→6 fucosyltransferase, galactosyltransferase (e.g., α1→3 galactosyltransferase, β1→4 galactosyltransferase or β1→3 galactosyltransferase) and a N-acetylglucosaminyltransferase (e.g., N-acetylglucosaminyltransferase I, II or III). In other embodiments, an additional agent can be used to increase incorporation of a chemical unit in a polysaccharide. For example, a monosaccharide can be peracetylated to increase diffusion of the monosaccharide into a cell, e.g., a recombinant cell. In other aspects, the agent can decrease or eliminate the presence of an enzyme present in the cell (e.g., UDP-N-acetylglucosamine-2-epimerase) such that increased incorporation of the monosaccharide units can occur.

In some embodiments, the modification is effected by directly modifying a polysaccharide moiety naturally present on the first, non-saccharide, moiety, thereby providing a modified second moiety. In other embodiments, the modification is effected by attaching a second polysaccharide moiety which differs from an existing polysaccharide naturally attached to said first moiety, e.g., by attaching a new or modified polysaccharide moiety to a first moiety that does not naturally include a second moiety, e.g., a first moiety in which a polysaccharide naturally attached to the first moiety has been removed, or a first moiety that does not normally have a polysaccharide attached to it. In other embodiments, the first moiety has an existing polysaccharide naturally attached to it removed, and a polysaccharide not naturally attached to it added as a modified second moiety, e.g., added at a position in the first moiety where the naturally existing polysaccharide had previously been attached or at a position in the first moiety where no naturally existing polysaccharide had previously been attached. In other embodiments, a second saccharide moiety is attached to a preselected site on a non-saccharide moiety. In other embodiments, additional saccharide moieties are attached to multiple sites on the non-saccharide moiety; the additional saccharide moieties may be chemically identical or different.

In some embodiments, the activity of the molecule is increased, decreased, eliminated by the modified second moiety. In one embodiment, the activity of the molecule is increased by the modified second moiety and the activity which is increased is selected from the group consisting of improved therapeutic index or activity after clinical administration, half-life, stability, IC₅₀ (ED₅₀), and binding. In another embodiment, the activity of the molecule is decreased or eliminated by the modified second moiety and the activity which is decreased or eliminated is a side effect associated with therapy, e.g., toxicity.

In some embodiments, the first moiety is a protein or fragment thereof and the modified second moiety is an N-linked polysaccharide, e.g., an N-linked polysaccharide selected from the group consisting of simple, complex, hybrid and high mannose polysaccharides. In another embodiment, the first moiety is a protein or fragment thereof and the modified second moiety is an O-linked polysaccharide. In yet another embodiment, the first moiety is a protein or fragment thereof and there are at least two or more modified second moieties associated with it, e.g., two or more N-linked polysaccharides, two or more O-linked polysaccharides, or combinations thereof. The protein or fragment thereof can be modified, e.g., by modifying the amino acid sequence to add a site for attaching the second moiety, e.g., the amino acid sequence of the protein or fragment thereof can be modified to replace an amino acid which does not serve as a site for attaching a polysaccharide or serves as a site for attaching a one type of polysaccharide (e.g., an O-linked polysaccharide) with another amino acid which serves as a site for attaching a different type of polysaccharide (e.g., an N-linked polysaccharide), or by adding to the amino acid sequence an additional amino acid which serves as a site for attaching a polysaccharide.

In other embodiments, the modified second moiety can be a glycosaminoglycan, or a Lewis sugar.

In some embodiments, the molecule is formed by attaching the first moiety and the modified second moiety by ligation, e.g., chemical, enzymatic or chemoenzymatic ligation.

In another embodiment, the modification includes purifying or enriching for one or more selected molecule species present in a preparation of molecules having a first and second moiety. In other words, the modification can be property of a collection of molecules, wherein the modification is not the introduction of a new second moiety but the alteration of the amounts or relative amounts of one or more species of a molecule having particular second moiety. E.g., one begins with a heterologous population of molecules, which are heterologous in the sense that the structure of the second moiety is heterologous, e.g., a population of a particular first moiety not all of which have the same second polysaccharide moiety. The structure of one or more of the heterologous second moiety species is determined. The modification can be effected by altering the structure of the second moiety or it can be effected by enriching for one or more of the heterologous second moiety species. By way of illustration, one can begin with preparation of a protein some of the protein molecules of which have a complex polysaccharide second moiety and some of which do not. The preparation is enriched for proteins having the complex structure of the second moiety.

In another aspect, the invention features molecules prepared by the methods described herein.

In another embodiment, the invention features a method for producing a molecule, e.g., a therapeutic molecule, which includes a first, non-saccharide moiety, e.g., a protein, polypeptide, peptide, amino acid or lipid, and a second, polysaccharide, moiety. The method includes: determining the chemical composition and structure of all or a portion of the second moiety, modifying the structure of the second moiety to provide a modified second moiety, evaluating or screening the molecule having the modified second moiety, e.g., for a biological activity or other chemical or physical property, and attaching the modified second moiety to a different first moiety.

In another aspect, the invention features a method of producing a first molecule which includes a first non-saccharide moiety and a second polysaccharide moiety. The method includes: selecting a modified second moiety which has been modified based upon its ability to confer a desired property on a second molecule, wherein the modified second moiety has been modified based upon its chemical structure; providing the modified second moiety which has been modified based upon its chemical structure and composition; and producing a first molecule which includes a first non-saccharide moiety and the modified second moiety, wherein the modified second moiety alters an activity of the first moiety, to thereby produce a first molecule.

As used herein, a non-saccharide moiety is a chemical moiety which includes a moiety which is other than a saccharide, for example, other than a di- or poly-saccharide. The most preferred non-saccharide moiety is a protein, polypeptide, peptide, amino acid, or lipid. The non-saccharide moiety may contain a saccharide component, for example, a glycoprotein can be a non-saccharide moiety, but as discussed above, the non-saccharide moiety must include an element which is not a saccharide.

### BRIEF DESCRIPTION OF THE DRAWINGS

FIG. 1 is a schematic of methods for rapid sequencing of carbohydrate structures.

FIGS. 2A and 2B are schematics of two techniques for synthesis of modified oligosaccharides. FIG. 2A shows automated solid phase synthesis, and FIG. 2B shows metabolic engineering in cell-based systems.

FIGS. 3A, 3B, 3C and 3D are a set of diagrams depicting notation schemes for branched chain analysis.

FIG. 4 measures the in vivo half-life of anti-MHC antibody (OKT3). 100 μg/kg of purified antibody, either with altered glycosylation or unaltered glycosylation, was injected intravenously into New Zealand rabbits. Blood samples were drawn at selected time points from 0-30 hours post-injection. Antibody levels were determined using an IgG-specific ELISA kit.

### DETAILED DESCRIPTION OF THE INVENTION

The invention is based, in part, on the discovery of rapid methods to produce and identify polysaccharides, and other sugar structures, in order to develop glycomolecules having altered activities for research and/or therapeutic purposes. The methods include the steps of determining the chemical composition and structure of a polysaccharide moiety, e.g., a polysaccharide moiety having a defined activity, to analyze the sequence of sugars on molecules such as proteins, polypeptides and lipids, modifying the chemical composition or structure of the polysaccharide moiety, using for example enzymatic or solid-phase methods, and screening the modified polysaccharide moiety as part of a glycomolecule, for optimized activity of the glycomolecule.

Polysaccharides

A polymer as used herein is a compound having a linear and/or branched backbone of chemical units which are secured together by linkages. In some, but not all, cases the backbone of the polymer may be branched. The term “backbone” is given its usual meaning in the field of polymer chemistry. A “polysaccharide” is a biopolymer comprised of linked saccharide or sugar units. In many polysaccharides, the basic building block of the polysaccharide is actually a disaccharide unit which can be repeating or non-repeating. Thus, a unit when used with respect to a polysaccharide refers to a basic building block of a polysaccharide and can include a monomeric building block (monosaccharide) or a dimeric building block (disaccharide). Chemical units of polysaccharides are much more complex than chemical units of other polymers such as nucleic acids and polypeptides. The polysaccharide unit has more variables in addition to its basic chemical structure than other chemical units. For example, the polysaccharide may be acetylated or sulfated at several sites on the chemical unit, or it may be charged or uncharged. In addition, different polysaccharides possess different monosaccharides connected by different glycosidic linkages, and may be branched or linear. Examples of monosaccharide chemical units include galactose, fucose, sialic acid, mannose, glucose, N-acetylglucosamine (GlcNAc), N-acetylgalactosamine (GalNAc), uronic acid (e.g., glucuronic acid and iduronic acid), xylose, as well as derivatives and analogs thereof.

A “plurality of chemical units” is at least two units linked to one another. A substituent, as used herein is an atom or group of atoms that substitute a unit, but are not themselves the units. As used herein with respect to linked units of a polymer, e.g., a polysaccharide, the two units are bound to each other by any physiochemical means. Any linkage, including covalent and non-covalent linkages, is embraced. Naturally occurring linkages are those ordinarily found in nature connecting chemical units of a particular polymer. The chemical units of a polymer can also be linked by synthetic or modified linkages.

The polymers may be native or naturally occurring polymers which occur in nature or non-naturally occurring polymers which do not exist in nature. The polymers can typically include at least a portion of a naturally occurring polymer. The polymers can be isolated or synthesized de novo. For example, the polymers can be isolated from natural sources, e.g., purified, as by cleavage and gel separation or may be synthesized e.g., by amplification in vitro, synthesized by chemical synthesis, or recombinantly produced, etc.

Methods of Determining Chemical Structure and Compositions of Polymers

It was discovered that specific chemical properties of a polysaccharide moiety of a molecule may be identified and manipulated in order to alter an activity, e.g., a therapeutic activity, or decrease or eliminate an activity, e.g., a negative side effect, of the molecule. In addition, the information obtained regarding the manipulated, i.e., modified, polysaccharide moiety can be applied to other molecules, e.g., other therapeutic molecules. For example, if a modified polysaccharide moiety is found to have an activity of interest, e.g., increased halflife of a molecule, that modified polysaccharide can be formulated (e.g., attached or synthesized) on a different molecule for which that activity, e.g., increased half-life is desired. Conversely, if a modified polysaccharide moiety or portion thereof is found to have an undesirable activity of interest, e.g., a negative side effect, that modified polysaccharide or portion thereof can be removed from a different molecule which has that undesirable side effect. The term “molecule” as used herein refers to proteins, polypeptides, peptides and lipids having a polysaccharide moiety associated with it.

The chemical properties of the polysaccharide may be modified by various techniques in order to alter an activity of active agents (e.g., a non-saccharide moiety of a molecule, e.g., a polypeptide or lipid) associated with the polysaccharide. In addition, the non-saccharide moiety can be associated with other polysaccharides in addition to at least one modified polysaccharide moiety. Methodologies have been developed to determine chemical signatures of polysaccharides. A chemical signature, as used herein, refers to information regarding, e.g., the identity, mass, charge and number of the mono- and di-saccharide building blocks of a polysaccharide and the core structure of a branched or unbranched polysaccharide, information regarding the physiochemical properties such as the overall charge (also referred to as the “net charge”), charge density, molecular size, charge to mass ratio, and sialic acid content as well as the relationships between the mono- and di-saccharide building blocks, e.g., linkages between chemical units of the polysaccharide, branch points, and active sites associated with these building blocks. Information regarding, e.g., the identity and number of mono- and di-saccharide building blocks, the core structure of a branched polysaccharide, the linkages between chemical units, branch points, sulfonation, sialylation, fucosylation, phosphorylation and acetylation, are considered properties of the chemical structure and composition of a polysaccharide. As used herein, a chemical signature may refer to all or part of a moiety. As described herein, it is possible to use specific chemical signatures such as the chemical structure and composition to modify polysaccharides in order to produce polysaccharide moieties which alter the activity of the molecules with which they are associated. The chemical signature can be provided by determining one or more primary outputs chosen from the following: the presence or the amount of one or more component saccharides or disaccharides; the presence or the amount of one or more block components, wherein a block component is one made up of more than one saccharides or polysaccharide, the presence of various linkages between chemical units, the presence of different branching structures of a polysaccharide; the presence or amount of one or more saccharide-representative, wherein a saccharide-representative is a saccharide modified to enhance detectability; the presence or amount of an indicator of three dimensional structure or a parameter related to three dimensional structure, e.g., activity, e.g., the presence or amount of a structure produced by cross-linking a polysaccharide, e.g., the cross-linking of specific saccharides which are not adjacent in the linear sequence; or the presence or amount of one or more modified saccharides, wherein a modified saccharide is one present in a starting material used to make a preparation but which is altered in the production of the preparation, e.g., a saccharide modified by cleavage. The chemical signature can also be provided by determining a secondary output, which include one or more of: total charge and density of charge.

Analysis of a polysaccharide moiety can be done by constructing a database containing known molecules having known properties, when analyzed using one or more techniques for analysis. A database allows for rapid analysis of polysaccharide moieties. For example, the known molecules may be saccharides, oligosaccharides or polysaccharides of known composition, structure and molecular mass. The properties may be the data obtained using a technique such as capillary or polyacrylamide gel electrophoresis, high pressure liquid chromatography (HPLC), gel permeation and/or ion exchange chromatography, nuclear magnetic resonance (NMR), mass spectrometry including electrospray or MALDI, modification with an enzyme such as digestion with an exoenzyme or endoenzyme, chemical digestion, or chemical modification. The property may also be measurement of a biological activity, such as the ability to inhibit coagulation, reaction or binding with an antibody, receptor or known ligand, or cleavage by an enzyme with known specificity. The process may be performed for the entire molecule or a portion thereof. The results may also be further quantitated.

Properties to be measured can include one or more of charge, molecular mass, nature and degree of sulfation, phosphorylation or acetylation, and type of saccharide. Additional properties include chirality, nature of substituents, quantity of substituents, molecular size, molecular length, composition ratios of substituents or units, type of basic building block of polysaccharide, hydrophobicity, enzymatic sensitivity, hydrophilicity, secondary structure and conformation (i.e. position of helicies), spatial distribution of substituents, linkages between chemical units, the number of branch points, core structure of a branched polysaccharide, ratio of one set of modifications to another set of modifications (i.e., relative amounts of acetylation or sulfation of various O-positions in sialic acid), and binding sites for proteins.

A property of a polymer may be identified by means known in the art. Molecular mass, for instance, may be determined by several methods including mass spectrometry. The use of mass spectrometry for determining the molecular mass of polymers is well known in the art. Mass spectrometry has been used as a powerful tool to characterize polymers because of its accuracy (±1 Dalton) in reporting the masses of fragments generated (e.g., by enzymatic cleavage), and also because only picomole sample amounts are required. For example, matrix-assisted laser desorption ionization mass spectrometry (MALDI-MS) has been described for identifying the molecular mass of polysaccharide fragments in publications such as Rhomberg, et al., PNAS USA 95, 4176-4181 (1998); Rhomberg, et al., PNAS USA 95, 12232-12237 (1998); and Ernst, et al. PNAS USA 95, 4182-4187 (1998). Other types of mass spectrometry known the art, such as electron spray-MS, fast atom bombardment mass spectrometry (FAB-MS), gas chromatography/mass spectrometry and collision-activated dissociation mass spectrometry (CAD) can also be used to identify the molecular mass of the polymer or polymer fragments. The compositional ratios of substituents or chemical units (quantity and type of total substituents or chemical units) may be determined using methodology known in the art, such as capillary electrophoresis. A polymer may be subjected to an experimental constraint such as enzymatic or chemical degradation to separate each of the chemical units of the polymers. These units then may be separated using capillary electrophoresis to determine the quantity and type of substituents or chemical units present in the polymer.

The mass spectrometry data may be a valuable tool to ascertain information about the polymer fragment sizes after the polymer has undergone degradation with enzymes or chemicals. After a molecular mass of a polymer is identified, it may be compared to molecular masses of other known polymers. Because masses obtained from the mass spectrometry data are accurate to one Dalton (1 Da), a size of one or more polymer fragments obtained by enzymatic digestion may be precisely determined, and a number of substituents (i.e., sulfates and acetate groups present) may be determined. One technique for comparing molecular masses is to generate a mass line and compare the molecular mass of the unknown polymer to the mass line to determine a subpopulation of polymers which have the same molecular mass. A “mass line” as used herein is an information database, preferably in the form of a graph or chart which stores information for each possible type of polymer having a unique sequence based on the molecular mass of the polymer. For instance, a mass line may be generated by uniquely assigning a particular mass to a particular length of a given fragment (all possible di, tetra, hexa, octa, up to a hexadecasaccharide), and tabulating the results. Methods of generating a database containing such information are provided below.

In addition to molecular mass, other properties may be determined using methods known in the art. The compositional ratios of substituents or chemical units (quantity and type of total substituents or chemical units) may be determined using methodology known in the art, such as capillary electrophoresis. A polymer may be subjected to an experimental constraint such as enzymatic or chemical degradation to separate each of the chemical units of the polymers. These units then may be separated using capillary electrophoresis to determine the quantity and type of substituents or chemical units present in the polymer. Additionally, a number of substituents or chemical units can be determined using calculations based on the molecular mass of the polymer.

In the method of capillary gel-electrophoresis, reaction samples may be analyzed by small-diameter, gel-filled capillaries. The small diameter of the capillaries (50 microns) allows for efficient dissipation of heat generated during electrophoresis. Thus, high field strengths can be used without excessive Joule heating (400 V/m), lowering the separation time to about 20 minutes per reaction run, therefore increasing resolution over conventional gel electrophoresis. Additionally, many capillaries may be analyzed in parallel, allowing amplification of generated polymer information.

The polymer can be further analyzed by applying experimental constraints to the polymer in a series of repetitions, where the constraints are different for each repetition. The experimental constraints may be any manipulation which alters the polymer in such a manner that it will be possible to derive structural information about the polymer or a unit of the polymer. In some embodiments, the experimental constraint applied to the polymer may be any one or more of the following constraints: enzymatic digestion, e.g., with an exoenzyme, an endoenzyme, a restriction endonuclease; chemical digestion; chemical modification; interaction with a binding compound; chemical peeling (i.e., removal of a monosaccharide unit); and enzymatic modification, for instance sulfation at a particular position with a sulfotransferase.

The structure and composition of the polysaccharide moiety can be analyzed, for example, by enzymatic degradation. For each type of monosaccharide and the various types of linkages between a particular monosaccharide and a polysaccharide chain, there exists a modifying enzyme. For example, galactosidases can be used to cleave glycosidic linkages after a galactose. Galactose can be present in a polysaccharide chain through an α1→3 glycosidic linkage or a β1→4 linkage. α-Galactosidase can be used to cleave α1→3 glycosidic linkages after a galactose and β-galactosidase can be used to cleave a β1→4 linkage after a galactose. Sources of β-galactosidase include S. pneumoniae. In addition, various sialidases can be used to specifically cleave an α2→3, an α2→6, an α2→8, or an α2→9 linkage after a sialic acid. For example, sialidase from A. urefaciens cleaves all sialic acids whereas other enzymes show a preference for linkage position. Sialidase (S. pneumoniae) cleaves α2→3 linkages almost exclusively whereas Sialidase II (C. perringens) cleaves α2→3 and α2→6 linkages only. Fucose can be linked to a polysaccharide by any of an α1→2, α1→3, α1→4, and α1→6 glycosidic linkage, and fucosidases which cleave each of these linkages after a fucose can be used. α-Fucosidase II (X. manihotis) cleaves only α1→2 linkages after fucose whereas α-fucosidase from bovine kidney cleaves only α1→6 linkages. GlcNAc can form three different types of linkages with a polysaccharide chain. These are a β1→2, a β1→4 and a β1→6 linkages. Various N-acetylglucosiaminidases can be used to cleave GlcNAc residues in a polysaccharide chain. β-N-Acetylhexosaminidase from Jack Bean can be used to cleave non-reducing terminal β1-2,3,4,6 linked N-acetylglucosamine, and N-acetylgalactosamine from oligosaccharides whereas alpha-N-Acetylgalactosaminidase (Chicken liver) cleaves terminal alpha 1→3 linked N-acetylgalactosamine from glycoproteins. Other enzymes such as aspartyl-N-acetylglucosaminidase can be used to cleave at a beta linkage after a GlcNAc in the core sequence of N-linked oligosaccharides.

Enzymes for degrading a polysaccharide at other specific monosaccharides such as mannose, glucose, xylose and N-acetylgalactosamine (GalNAc) are also known.

Degrading enzymes are also available which can be used to determine branching identity, i.e., is a polysaccharide mono-, bi-, tri- or tetrantennary. Various endoglycans are available which cleave polysaccharides having a certain number of branches but do not cleave polysaccharides having a different number of branches. For example, EndoF2 is an endoglycan that clips only biantennary structures. Thus, it can be used to distinguish biantennary structures from tri- and tetrantennary structures.

In addition, modifying enzymes can be used to determine the presence and number of substitutents of a chemical unit. For example, enzymes can be used to determine the absence or presence of sulfates using, e.g., a sulfatase to remove a sulfate group or a sulfatransferase to add a sulfate group.

Glucuronidase and iduronidase can also be used to cleave at the glycosidic linkages after a glucuronic acid and an iduronic acid, respectively. In a similar manner, enzymes exist that cleave galactose residues in a linkage specific manner and enzymes that cleave mannose residues in a linkage specific manner.

The property of the polymer that is detected by this method may be any structural property of a polymer or unit. For instance, the property of the polymer may be the molecular mass or length of the polymer. In other embodiments the property may be the compositional ratios of substituents or units, type of basic building block of a polysaccharide, hydrophobicity, enzymatic sensitivity, hydrophilicity, secondary structure and conformation (i.e., position of helices), spatial distribution of substituents, linkages between chemical units, number of branch points, core structure of a branched polysaccharide, ratio of one set of modifications to another set of modifications (i.e., relative amounts of sulfation, actylatiion or phosphorylation at the position for each), and binding sites for proteins.

Methods of identifying other types of properties may be easily identifiable to those of skill in the art and may depend on the type of property and the type of polymer. For example, hydrophobicity may be determined using reverse-phase high-pressure liquid chromatography (RP-HPLC). Enzymatic sensitivity may be identified by exposing the polymer to an enzyme and determining a number of fragments present after such exposure. The chirality may be determined using circular dichroism. Protein binding sites may be determined by mass spectrometry, isothermal calorimetry and NMR. Linkages may be determined using NMR and/or capillary electrophoresis. Enzymatic modification (not degradation) may be determined in a similar manner as enzymatic degradation, i.e., by exposing a substrate to the enzyme and using MALDI-MS to determine if the substrate is modified. For example, a sulfotransferase may transfer a sulfate group to an oligosaccharide chain having a concomitant increase of 80 Da. Conformation may be determined by modeling and nuclear magnetic resonance (NMR). The relative amounts of sulfation may be determined by compositional analysis or approximately determined by raman spectroscopy.

Methods for identifying the charge and other properties of polysaccharides have been described in Venkataraman, G., et al., Science, 286, 537-542 (1999), and U.S. patent application Ser. Nos. 09/557,997 and 09/558,137, both filed on Apr. 24, 2000, which are hereby incorporated by reference. Other suitable methods for use as described here are known to those skilled in the art. See, for example, Keiser, et al., Nature Medicine 7(1), 1-6 (January 2001); Venkataraman, et al., Science 286, 537-542 (1999). See also, U.S. Pat. No. 6,190,522 to Haro, U.S. Pat. No. 5,340,453 to Jackson, and U.S. Pat. No. 6,048,707 to Klock, for specific techniques that can be utilized.

In addition to being useful for identifying a property, compositional analysis, as described above, also may be used to determine a presence and composition of an impurity as well as a main property of the polymer. Such determinations may be accomplished if the impurity does not contain an identical composition as the polymer. To determine whether an impurity is present may involve accurately integrating an area under each peak that appears in the electrophoretogram and normalizing the peaks to the smallest of the major peaks. The sum of the normalized peaks should be equal to one or close to being equal to one. If it is not, then one or more impurities are present. Impurities even may be detected in unknown samples if at least one of the disaccharide units of the impurity differs from any disaccharide unit of the unknown. If an impurity is present, one or more aspects of a composition of the components may be determined using capillary electrophoresis.

Database for Determining Chemical Structure and Composition of a Polymer

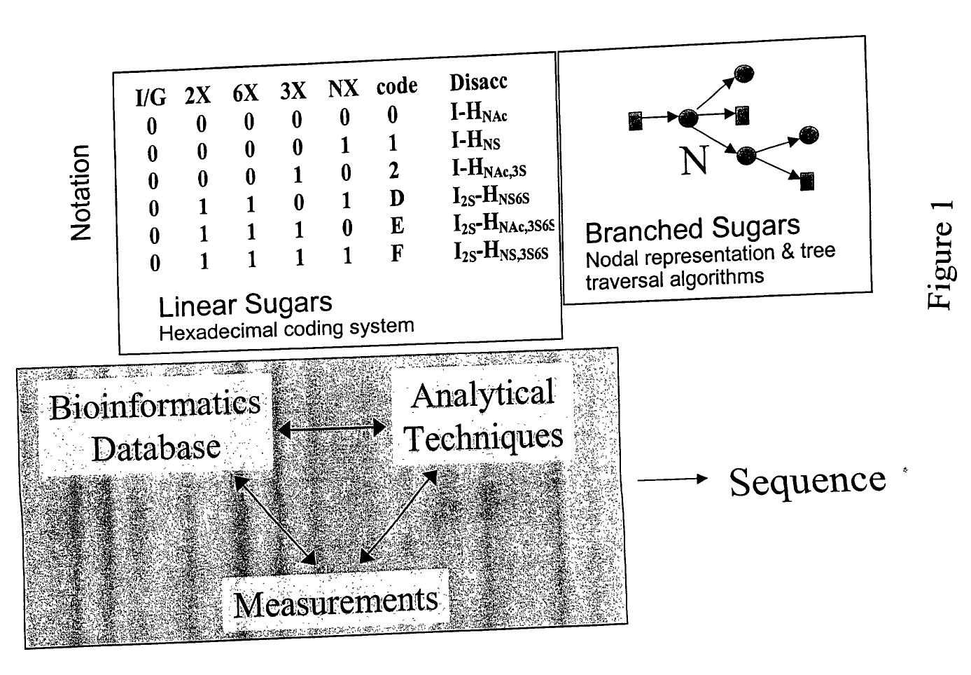

The data obtained using these methods can be analyzed and put into a database (see FIG. 1). A “database”, as used herein, refers to a repository of one or more structures or representatives (unique signatures) of the structure or structures, e.g., mass, charge, mass-to-charge, to which one or more unknown polysaccharides are compared. The database can be, for example, a flat file, a relational database, a table, an object or structure in a computer readable volatile or non-volatile memory, or any data accessible by computer program. Once the database has been constructed, the polysaccharide moiety to be characterized, or a portion thereof, can be analyzed, and the results inputted into a computer for comparison with the known polysaccharide molecules in the database. Additional tests can be conducted based on those results, and then, if necessary, the process can be repeated until the polysaccharide has been identified. For example, the structure and composition of a polysaccharide can be determined by comparing the length and/or molecular mass of the polysaccharide moiety to a database of polysaccharides having a known length and/or molecular mass. A subpopulation of polysaccharides having the same length and/or a similar molecular mass as the polysaccharide moiety can be selected. An experimental constraint can be applied to the polysaccharide moiety to determine a property of the polysaccharide moiety and polysaccharides of the subpopulation which do not have the same property when the same experimental constraint has been applied to them can be eliminated. Additional experimental constraints can be applied and additional polysaccharides of the subpopulation can be eliminated based on the results obtained using those additional constraints until the polysaccharide moiety is identified.

A database can be constructed to analyze branched or unbranched polymers, e.g., branched or unbranched polysaccharides.

Branched polysaccharides include a few building blocks, chemical units, that can be combined in several different ways, thereby, coding for many sequences. For instance, a trisaccharide, in theory, can give rise to over 6 million different sequences. The methods for analyzing branched polysaccharides, in particular, are advanced by the creation of an efficient nomenclature that is amenable to computational manipulation. Thus, an efficient nomenclature for branched sugars is useful for determining the structure and composition of polysaccharide moieties. The following are two types of numerical schemes that may be used to encode the sequence information of branched polysaccharides. These have been developed in order to bridge the widely used graphic (pictorial) representation and the proposed numerical scheme discussed below.

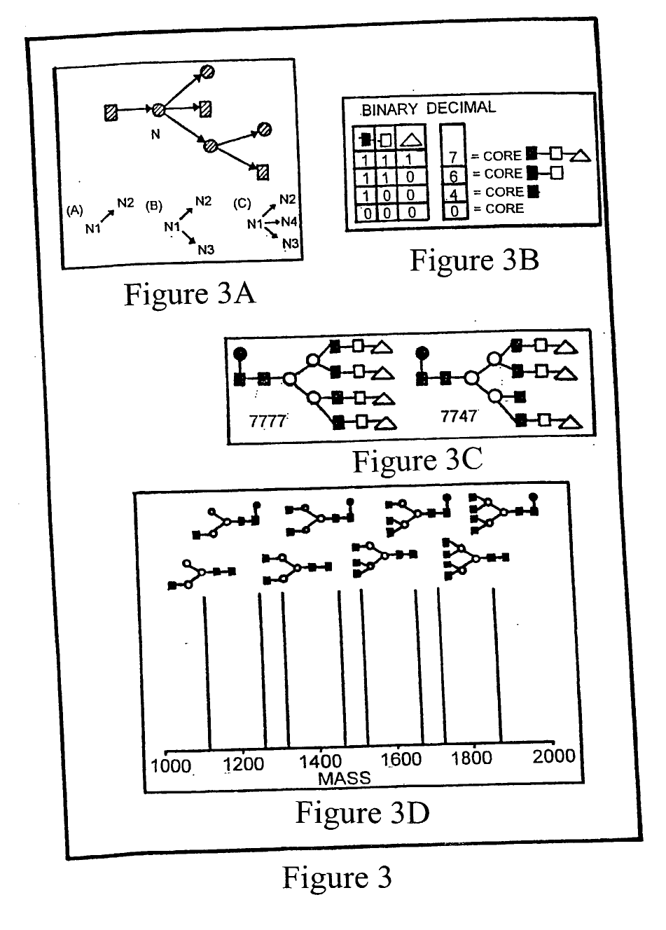

The first notational scheme is a byte-based (binary-scheme) notation scheme. This notation scheme is based on a binary numerical system. The binary representation in conjunction with a tree-traversing algorithm can be used to represent all the possible combinations of the branched polysaccharides. The nodes (branch points) are easily amenable to computational searching through tree-traversing algorithms (FIG. 3A). FIG. 3A shows a notation scheme for branched sugars. Each monosaccharide unit can be represented as a node (N) in a tree. The building blocks can be defined as either (A), or (B), or (C) where N 1, N2, N3, and N4 are individual monosaccharides. Each of these combinations can be coded numerically to represent building blocks of information. By defining glycosylation patterns in this way, there are several tree traversal and searching algorithms in computer science that may be applied to solve this problem.

A simpler version of this notational scheme is shown in FIG. 3B. This simplified version may be extended to include all other possible modifications including unusual structures. For examples, an N-linked glycosylation in vertebrates contains a core region (the tri-mannosyl chitobiose moiety), and up to four branched chains from the core. In addition to the branched chains, the notation scheme also includes other modification (such as addition of fucose to the core, or fucosylation of the GlcNAc in the branches or sialic acid on the branches). Thus, the superfamily of N-linked polysaccharides can be broadly represented by three modular units: a) core region: regular, fucosylated and/or bisected with a GlcNAc, b) number of branches: up to four branched chains (e.g., biantennary, triantennary, tetrantennary), each with GlcNAc, Gal and Neu, and c) modifications of the branch sugars. These modular units may be systematically combined to generate all possible combinations of the polysaccharide. Representation of the branches and the sequences within the branches can be performed as a n-bit binary code (0 and 1) where n is the number of monosaccharides in the branch. FIG. 3C depicts a binary code containing the entire information regarding the branch. Since there are up to four branches possible, each branch can be represented by a 3-bit binary code, giving a total of 12 binary bits. The first bit represents the presence (binary 1) or absence (binary 0) of the GlcNAc residue adjoining the mannose. The second and the third bit similarly represent the presence or absence of the Gal and the Neu residues in the branch. Hence a complete chain containing GlcNAc-Gal-Neu is represented as binary (111) which is equivalent to decimal 7. Four of the branches can then be represented by a 4 bit decimal code, the first bit of the decimal code for the first branch and the second, the second branch etc. (right).

This simple binary code does not contain the information regarding the linkage (a vs. β and the 1-6 or 1-3, etc.) to the core. This type of notation scheme, however, may be easily expanded to include additional bits for branch modification. For instance, the presence of a 2-6 branched neuraminic acid (Neu) to the GlcNAc in the branch can be encoded by a binary bit.

The second notational scheme that can be used is a prime decimal notation scheme. Similar to the binary notation described above, a second computationally friendly numerical system, which involves the use of a prime number scheme, has been developed. The algebra of prime numbers is extensively used in areas of encoding, cryptography and computational data manipulations. The scheme is based on the theorem that for small numbers, there exists a uniquely definable set of prime divisors. In this way, composition information may be rapidly and accurately analyzed.

This scheme can be illustrated by the following example. The prime numbers 2, 3, 5, 7, 11, 13, 17, 19, and 23 are assigned to nine common building blocks of polysaccharides. The composition of a polysaccharide chain may then be represented as the product of the prime decimals that represent each of the building blocks. For illustration, GlcNAc is assigned the number 3 and mannose the number 2. The core is represented in this scheme as 2×2×2×3×3=72 (3 mannose and 2 GlcNAcs). This notation, therefore, relies on the mathematical principle that 72 can be only expressed as the combination of three 2s and two 3s. The prime divisors are therefore unique and can encode the composition information.

From this number, the mass of the polysaccharide chain can be determined. The power of the computational approaches of the notional scheme may be used to systematically develop an exhaustive list of all possible combinations of the polysaccharide sequences. For instance, an unconstrained combinatorial list of possible sequences of size mⁿ, where m is the number of building blocks and n is the number of positions in the chain may be used. In FIG. 3C, there are 256 different saccharide combinations that are theoretically possible (4 combinations for each branch and 4 branches=4⁴).

A mass line of the 256 different polysaccharide structures may be plotted. Then, the rules of biosynthetic pathways may be used to further analyze the polysaccharide. In the example (shown in FIG. 3B), it is known that the first step of the biosynthetic pathway is the addition of GlcNAc at the 1-3) linked chain (branch 1). Thus, branch 1 should be present for any of the other branches to exist. Based on this rule, the 256 possible combinations may be reduced using a factorial approach to conclude that the branch 2, 3, and 4 exist if and only if branch one is non-zero. Similar constraints can be incorporated at the notation level before generation of the master list of ensembles. With the notation scheme in place, experimental data can be generated (such as MALDI-MS or CE or chromatography) and those sequences that do not satisfy this data can be eliminated. An iterative procedure therefore enables a rapid convergence to a solution.

To identify branching patterns, a combination of MALDI-MS and CE (or other techniques) can be used. Elimination of the pendant arms of the branched polysaccharide may be achieved by the judicious use of exo and endoenzymes. All antennary groups may be removed, retaining only the GlcNAc moieties extending from the mannose core and forming an “extended” core. In this way, information about branching is retained, but separation and identification of glycoforms is made simpler. One methodology that could be employed to form extended cores for most polysaccharide structures is the following. Addition of sialidases, and fucosidases will remove capping and branching groups from the arms. Then application of endo-β-galactosidase will cleave the arms to the extended core. For more unusual structures, other exoglycosidases are available, for instance xylases and glucosidases. By addition of a cocktail of degradation enzymes, any polysaccharide motif may be reduced to its corresponding “extended” core. Examples of degradation enzymes which can be used include galactosidase (e.g., α-galactosidase or β-galactosidase), sialidase, fucosidase, and acetylglucosaminidase. Identification of “extended” core structures can be made by mass spectral analysis. There are unique mass signatures associated with an extended core motif depending on the number of pendant arms (FIG. 3D). FIG. 3D shows a massline of the “extended” core motifs generated upon exhaustive digest of glycan structures by the enzyme cocktail. Shown are the expected masses of mono-, di-, tri- and tetrantennary structures both with and without a fucose linked α1→6 to the core GlcNAc moiety (from left to right). All of the “extended” core structures have a unique mass signature that can be resolved by MALDI-MS (from left to right). Quantification of the various glycan cores present may be completed by capillary electrophoresis, which has proven to be a highly rapid and sensitive means for quantifying polysaccharide structures. See, e.g., Kakehi, K. and S. Honda, Analysis of glycoproteins, glycopeptides and glycoprotein-derived polysaccharides by high-performance capillary electrophoresis. J Chromatogr A, 1996. 720(1-2):377-393.

Methods for Synthesis or Production of Modified Molecules

Once the starting material has been characterized, and the desired components of the polysaccharide moiety identified, the modified polysaccharide can be produced.

The method for modifying the polysaccharide can be determined, e.g., based upon the information obtained regarding the chemical signature of the polysaccharide. For instance, based upon the structure and composition of the desired polysaccharide and the nature of the modification, the polysaccharide can be synthesized, e.g., by enzymatic modification or can be produced by recombinant organisms, e.g., by controlling degradation. In other aspects, the modified polysaccharide can be obtained, e.g., by SAR-based purification methods to obtain a selected polysaccharide to provide an altered activity to a non-saccharide moiety.

Enzymatic modification of a polysaccharide moiety can be obtained, e.g., by removing and/or adding select monosaccharides from the polysaccharide. For instance, an enzyme which selectively cleaves a polysaccharide can be used to modify the polysaccharide moiety. Examples of degrading enzymes which can be used include α-galactosidase to cleave a α1→3 glycosidic linkage after a galactose, β-galactosidase to cleave a β1→4 linkage after a galactose, an α2→3 sialidase to cleave a α2→3 glycosidic linkage after a sialic acid, an α2→6 sialidase to cleave after an α2→6 linkage after a sialic acid, an α1→2 fucosidase to cleave a α1→2 glycosidic linkage after a fucose, a α1→3 fucosidase to cleave a α1→3 glycosidic linkage after a fucose, an α1→4 fucosidase to cleave a α1→4 glycosidic linkage after a fucose, an α1→6 fucosidase to cleave an α1→6 glycosidic linkage after a fucose. β-N-Acetylhexosaminidase from Jack Bean can be used to cleave non-reducing terminal β1→2,3,4,6 linked N-acetylglucosamine, and N-acetylgalactosamine from oligosaccharides whereas alpha-N-Acetylgalactosaminidase (Chicken liver) cleaves terminal alpha 1→3 linked N-acetylgalactosamine from glycoproteins. Other enzymes such as aspartyl-N-acetylglucosaminidase can be used to cleave at a beta linkage after a GlcNAc in the core sequence of N-linked oligosaccharides.

In addition, glucuronidase and iduronidase can be used to cleave at the glycosidic linkages after a glucuronic acid and an iduronic acid, respectively.

By selective cleavage, a modified polysaccharide can be generated such that, e.g., chemical units or regions of the polysaccharide which are not involved and/or do not influence a desired biological activity can be cleaved, and regions of the polysaccharide which are involved and/or influence a biological activity remain intact. As used herein, the term “intact” means uncleaved and complete.

Enzymatic modification can also be used to add monosaccharides to the polysaccharide. Monosaccharides added to a polysaccharide chain can be incorporated in activated form. Activated monosaccharides, which can be added, include UDP-galactose, UDP-glucose, UDP-N-acetylglucosamine, UDP-N-acetylgactosamine, UDP-Glucuronic acid, UDP-Iduronic acid, UDP-xylose, GDP-mannose, GDP-fucose and CMP-sialic acid. Activated forms of monosaccharides can be generated by methods known in the art. For example, galactose can be activated to UDP-galactose by several ways including: direct phosphorylation at the 1-position to give Gal-1-P, which can react with UTP to give UDP-galactose; Gal-1-P can be converted to UDP-galactose via uridyl transferase exchange reaction with UDP-glucose that displaces Glc-1-P. UDP-glucose can be derived from glucose by converting glucose to Gle-6-P by hexokinase and then either to Fru-6-P by phosphoglucose isomerase or to Glc-1-P by phosphoglucomutase. Reaction of Glc-1-P with UTP forms UDP-glucose. GDP-fucose can be derived from GDP-Man by reduction with CH₂OH at the C-6 position of mannose to a CH₃. This can be done by the sequential action of two enzymes. First, the C-4 mannose of GDP-Man is oxidized to a ketone, GDP-4-dehydro-6-deoxy-mannose, by GDP-Man 4,6-dehydratase along with reduction of NADP to NADPH. The GDP-4-keto-6-deoxymannose is the epimerized at C-3 and C-5 to form GDP4-keto-6-deoxyglucose and then reduced with NADPH at C-4 to form GDP-fucose. Methods of obtaining other activated monosaccharide forms can be found in, e.g., Varki, A et al., eds., Essentials of Glycobiology, Cold Spring Harbor Press, Cold Spring Harbor, N.Y. (1999).

An activated monosaccharide can be incorporated into a polysaccharide chain using the appropriate glycosyltransferase. For example, to incorporate a sialic acid, CMP-sialic acid onto a polysaccharide chain, a sialyltransferase, e.g., α2→3 sialyltransferase or α2→6 sialyltransferase, can be used. To incorporate a fucose, a fucosyltransferase, e.g., α1→2 fucosyltransferse, α1→3 fucosyltransferase, α1→4 fucosyltransferase or α1→6 fucosyltransferase, can be used. Glycosyltransferases for incorporating galactose and GlcNAc include a galactosyltransferase (e.g., α1→3 galactosyltransferase, β1→4 galactosyltransferase or β1→3 galactosyltransferase) and a N-acetylglucosaminyltransferase (e.g., N-acetylglucosaminyltransferase I, II or III), respectively. Glycosyltransferases for incorporating other monsaccharides are known.

Enzymatic modification of a polysaccharide can also include both removal of one or more monosaccharide units and then addition of one or more different monosaccharide units to obtain the desired modified polysaccharide.

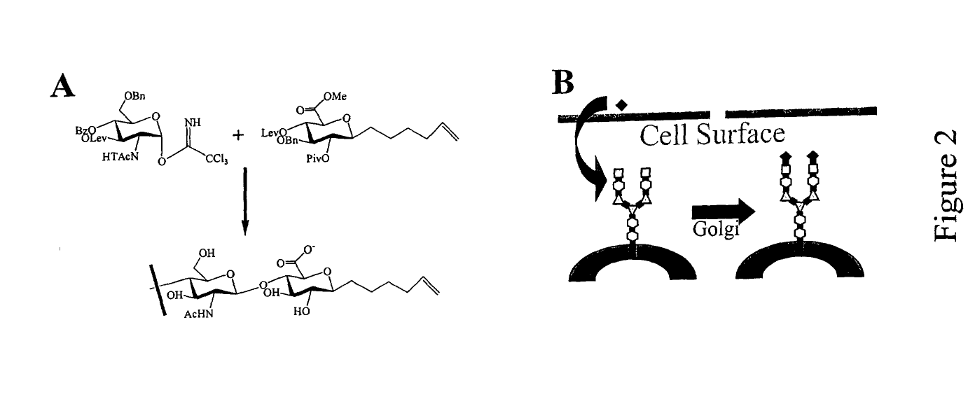

Methods for synthesis using enzymes such as glycosyltransferases are described by Bowman, et al., Biochemistry 40(18):5382-5391 (2001). See also FIG. 2B. Examples of enzymatic synthesis of oligosaccharides are also described in U.S. Pat. No. 6,030,815. U.S. Pat. No. 5,945,322 describes glycosyltransferases for the biosynthesis of oligosaccharides, and genes encoding them. N-containing saccharides and method for the synthesis of N-containing saccharides from amino-deoxy-disaccharides and amino-deoxyoligosaccharides are described in U.S. Pat. No. 5,856,143. Sialyltransferases are described in U.S. Pat. No. 6,280,989. Keratan sulfate oligosaccharide fraction and pharmaceutical containing the oligosaccharide are described in U.S. Pat. No. 6,159,954.

The methods for synthesis of saccharides include enzymatic as well as chemical synthesis. An example of an automated solid-support synthesis of an oligosaccharide is described by Hewitt and Seeberger, J. Org. Chem. 15:66(12):4233-4243 (June 2001) and Plante, et al., Science 23:291(5508):1523-1527 (2001). This method relies on assembly from monosaccharide units using a solid-phase synthesizer. A branched dodecasaccharide is synthesized through the use of glycosyl phosphate building blocks and an octenediol functionalized resin. The oligosaccharide is then cleaved from the support. See also, Org. Lett. 2(24):3841-3843 (2000); Andrade, et al., Org. Lett. 1(11): 1811-1814 (1999). See further FIG. 2A. An apparatus for the synthesis of saccharide compositions is described in U.S. Pat. No. 6,156,547.

In addition, other saccharides can be synthesized. For instance, lactosamine oligosaccharides and methods for producing lactosamine oligosaccharides are described in U.S. Pat. No. 6,132,994. A lactosamine saccharide can be added to a polysaccharide chain.

Methods for saccharide characterization and sequencing of oligosaccharides, and methods for reagent array-electrochemical detection are described in U.S. Pat. No. 5,753,454. Methods of sequencing of oligosaccharides are described in U.S. Pat. No. 5,667,984. Methods for determining sugar chain structure are described in U.S. Pat. No. 5,500,342. A process for characterizing the glycosylation of glycoproteins and for the in vitro determination of the bioavailability of glycoproteins are described in U.S. Pat. No. 6,096,555.

Oligosaccharide analogs can also be added to a polysaccharide. Methods for synthesis of oligosaccharide analogs are well known to those skilled in the art. In general, there are considered nine naturally occurring monosaccharides: glucose, xylose, fucose, mannose, N-acetyl galactosamine, N-acetyl glucosamine, galactose, ribose and sialic acid. Any non-natural analogues of these can be added to the glycoproteins. Derivatives, or analogs, of other monosaccharides, i.e. hexose and/or pentose, may be used. Nonlimiting examples include: amidine, amidrazone and amidoxime derivatives of monosaccharides (U.S. Pat. No. 5,663,355, hereby incorporated by reference), 1,3,4,6-tetra-O-acetyl-N-acylmannosamine or derivative thereof, analogs or derivatives of sugars or amino sugars having 5 or 6 carbons in the glycosyl ring; including aldoses, deoxyaldoses and ketoses without regard for orientation or configuration of the bonds of the asymmetric carbons. This includes such sugars as ribose, arabinose, xylose, lyxose, allose, altrose, glucose, idose, galactose, talose, ribulose, xylulose, psicose, N9 acetylglucosamine, .N-acetylgalactosamine, N-acetylmannosamine, N-acetylneuraminic acid, fructose, sorbose, tagatose, rhamnose and fucose. Exemplary monosaccharide analogs and derivatives derived from Glc, GlcNAc, Gal, GalNAc, Man, Fuc, and NeuAc as taught in U.S. Pat. No. 5,759,823; hereby incorporated by reference, can be used.

Sialic acids represent the most abundant terminal sugar components on mammalian glycoproteins. Sialic acid/fucose-based pharmaceutical compositions are described in U.S. Pat. No. 5,679,321. Methods for making synthetic ganglioside derivatives are described in U.S. Pat. No. 5,567,684. Bivalent sialyl-derivatized saccharides are described in U.S. Pat. No. 5,559,103. Derivatives and analogues of 2-deoxy-2,3-didehydro-N acetyl neuraminic acid and their use as antiviral agents are reported in U.S. Pat. No. 5,360,817. Examples of preferred sugar monosaccharide analogs include those that functionally mimic sialic acid, but are not recognized by endogenous host cell sialylases. Sialyltransferases and other enzymes that are involved in sialic acid metabolism often recognize “unnatural” or “modified” monosaccharide substrates (Rosa et al., Biochem. Biophys. Res. Commun., 190, 914, 1993; Fitz and Wong, J., Org. Chem., 59, 8279, 1994; Shames et al., Glycobiology, 1, 187, 1991; Sparks et al., Tetrahedron, 49, 1, 1993; Lin et al., J. Am. Chem. Sot., 114, 10138, 1992). It has been clearly demonstrated that mannosamine derivatives are converted to sialic acid analogs and incorporated into glycoproteins in cell culture and in rats. In these studies N-acetylmannosamine (ManNAc), the six carbon precursor for sialic acid, was used as a substrate for the synthesis of metabolically modified glycoproteins, wherein the N-acetyl group of ManNAc was substituted with N-propanoyl, N-butanoyl, or N-pentanoyl (Keppler et al., J. Biol. Chem., 1995, 270,3:1308-1314; and Varki A., J. FASEB, 1991, 2:226-235). Examples of sugar monosaccharide analogs that may also be used include, but are not limited to, N-levulinoyl mannosamine (ManLev), Neu5Acα-methyl glycoside, 10 Neu5Acα-methyl glycoside, Neu5Acα-benzyl glycoside, Neu5Aα-benzyl glycoside, Neu5Acα-methylglycoside methyl ester, Neu5Acα-methyl ester, 9-O-Acetyl-N-acetylneuraminic acid, 9-O-Lactyl-N-acetylneuraminic acid, N-azidoacetylmannosamine and O-acetylated variations thereof, and Neu5Acα-ethyl thioglycoside. Examples of sialic acid analogs and methods that may be used to produce such analogs are taught in U.S. Pat. No. 5,759,823 and U.S. Pat. No. 5,712,254; hereby incorporated by reference.

Oligosaccharides can also be produced in recombinant systems, although this is typically during glycoprotein production. Methods of controlling the degradation of glycoprotein oligosaccharides produced by cultured CHO cells is described by U.S. Pat. No. 5,510,261; methods for controlling sialic acid derivatives in the production of recombinant glycoproteins is described in U.S. Pat. No. 5,459,031. Compounds for altering cell surface sialic acids and methods of use thereof are disclosed in U.S. Pat. No. 6,274,568; methods for sialylation of N-linked glycoproteins expressed in baculovirus expression systems are described in U.S. Pat. No. 6,261,805.

In some aspects, the glycoprotein can be a recombinant glycoprotein produced in a genetically engineered host, either an animal or yeast, fungi, plants, or other eukaryotic cell expression system, although glycoproteins which are normally expressed by the cells can also be modified with non-naturally occurring saccharides. In another embodiment, the non-naturally occurring saccharides are added to the isolated or synthetically produced glycoproteins, by providing the requisite enzymes in combination with the non-naturally occurring substrates, either in a cellbased system or in a cell-free system. The glycoproteins can be modified initially using enzymes to remove all or part of the saccharides, then the non-naturally occurring saccharides added. In yet another embodiment, the starting material may be a protein produced, for example, in a bacterial system wherein the protein is not glycosylated. The protein can then be modified as described above, to produce a glycoprotein including non-naturally occurring saccharides.

These methods can make use of monosaccharide substrates that are taken up by a host cell, converted to “activated” monosaccharide substrates in uivo and incorporated into the recombinantly expressed protein via the biosynthetic machinery endogenous to the host cell. The protein may be modified by the addition of any monosaccharide, or derivative thereof, that is added to the cell culture, fed to the host animal, and taken up by the host cell where it is attached to the glycoprotein, or which is added to the glycoprotein in a cell-free medium by enzyme(s). The methods are amenable to any host cell which can be manipulated to produce a modified glycoprotein. The host cell uses endogenous biochemical processing pathways to convert, or process, the exogenously added monosaccharide into an activated form that serves as a substrate for conjugation to a target glycoprotein in vivo or in vitro.

The method for altering the glycosylation of a polysaccharide moiety associated with a protein can includes the following steps: a) contacting a host cell producing the protein to be modified, with a monosaccharide derivative, or analog; and b) incubating the cell under conditions whereby the cell (i) internalizes the monosaccharide derivative, or analog, (ii) biochemically processes the monosaccharide derivative, or analog, and (iii) conjugates the processed monosaccharide derivative, or analog, to an expressed target glycoprotein. The saccharides are added in or administered to a concentration range between 1 micromolar and 100 millimolar, over the course of glycoprotein production or when there is a change in media, depending on culture conditions.

In an in vitro system, the enzymes required for activation and attachment of the saccharides are added to the protein, in the same concentration ranges. The enzymes can be in purified or only partially purified form. Examples of such enzymes are provided herein.

Various systems are available for making these glycoproteins. For example, the glycoproteins can be produced in a cell-based expression system or in a cell-free system. The former is preferred. Cells can be eukaryotic or procaryotic, as long as the cells provide or have added to them the enzymes to activate and attach the non-natural saccharides and the non-natural saccharides are present in the cell culture medium or fed to the organism including the cells. Examples of eukaryotic cells include yeast, insect, fungi, plant and animal cells, especially mammalian cells, most particularly cells that are maintained in culture such as CHO cells and Green Monkey cells. These organisms all normally glycosylate proteins, although not necessarily in the same manner or with the same saccharides. In the most preferred embodiment, the cells are mammalian. The eukaryotic cells may also be organisms such as animals, where the non-natural saccharides are provided to the animal typically by feeding. In another preferred embodiment, cell lines having genetically modified glycosylation pathways that allow them to carry out a sequence of enzymatic reactions, which mimic the processing of glycoproteins in humans, may also be used.

Currently available systems include but are not limited to: mammalian cells such as Chinese hamster ovary cells (CHO), mouse fibroblast cells, mouse myeloma cells (Arzneimittelforschung. 1998 August; 48(8): 870-880), Jurkat cells, HL-60 and HeLa cells; transgenic animals such as goats, sheep, mice and others (Dente Prog. Clin. Biol. 1989 Res. 300: 85-98, Ruther et al., 1988 Cell 53(6): 847-856; Ware, J., et al. 1993 Thrombosis and Haemostasis 69(6): 1194-1194; Cole, E. S., et al. 1994 J. CeZZ. Biochem. 265-265); plants (for example, Arabidopsis thaliana, rape seed, corn, wheat, rice, tobacco etc.) (Staub, et al. 2000 Nature Biotechnology 1S(3): 333-338)(McGarvey, P. B., et al. 1995 Bio-Technology 13(13): 1484-1487; Bardor, M., et al. 1999 Trends in Plant Science 4(9): 376-380); insect cells (for example, Spodoptera frugiperda Sf9, Sf21, Trichoplusia ni, etc. in combination with recombinant baculoviruses such as Autographa californica multiple nuclear polyhedrosis virus which infects lepidopteran cells)(Altmans et al., 1999 Glycoconj. J. 16(2): 109-123); bacteria, including species such as Escherichia coli commonly used to produce recombinant proteins; various yeasts and fungi such as Pichiapastoris, Pichia methanolica, Hansenula polymorpha, and Saccharomyces cerevisiae which have been particularly useful as eukaryotic expression systems, since they are able to grow to high cell densities and/or secrete large quantities of recombinant protein.

Methods of transfecting cells, and reagents such as promoters, markers, signal seqeucnes which can be used for recombinant expression are known.

Non-Saccharide Molecules

The methods described herein can be used to modify a polysaccharide composition naturally associated with a non-saccharide moiety or can be used to add a polysaccharide to a non-saccharide moiety that is not naturally associated with the polysaccharide. In this regard, the non-saccharide moiety can be one that is naturally associated with a different polysaccharide moiety (e.g., where a polysaccharide naturally associated with the non-saccharide moiety is replaced with a polysaccharide which is not naturally associated with the non-saccharide moiety) or the non-saccharide moiety can be one that is not naturally associated with any polysaccharide moiety. In other aspects, the polysaccharide moiety can be associated with a non-saccharide moiety at a position in the non-saccharide moiety which is not naturally associated with a polysaccharide. In some embodiments, the non-saccharide moiety can be associated with more than one polysaccharide and at least one or more of those polysaccharides is modified. In other aspects, the non-saccharide moiety can be associated with one or more polysaccharides, and at least one additional polysaccharide moiety is added, e.g., at a position in the non-saccharide moiety that is not naturally associated with a polysaccharide. In yet other embodiments, the non-saccharide moiety can be naturally associated with more than one polysaccharide, at least one of which has been modified by the methods disclosed herein. In addition, the non-saccharide moiety can have at least one additional polysaccharide added, e.g., at a position in the non-saccharide moiety that is not naturally associated with a polysaccharide.

Examples of non-saccharide molecules include, but are not limited to, proteins, polypeptides, peptides, amino acids, lipids, and heterogeneous mixtures thereof.

Proteins or fragments thereof can be associated with one or more modified polysaccharide to form a glycoprotein or glycopolypeptide using the methods disclosed herein. Examples of classes of proteins which can be used as the non-saccharide portion of a molecule include antibodies, enzymes, growth factors, cytokines and chemokines. Antibodies which can be associated with a modified polysaccharide, as described herein, include CDP-571, gemtuzumab ozogamicin, biciromab, imciromab, capromab, ¹¹¹indium satumomab pendetide, bevacizumab, ibritumomab tiuxetan, cetuximab, sulesomab, afelimomab, HuMax-CD4, MDX-RA, palivizumab, basiliximab, inolimomab, lerdelimumab, pemtumomab, idiotypic vaccine (CEA), Titan, Leucotropin, etanercept, pexelizumab, alemtuzumab, natalizumab, efalizumab, trastuzumab, epratuzumab, palivizumab, daclizumab, lintuzumab, Cytogam, Engerix-B, Enbrel, Gamimune (IgG), Meningitec, Rituxan, Synagis, Reopro, Herceptin, Sandoglobulin, Menjugate, and BMS-188667. Growth factors, enzymes and receptors which can be used as non-saccharide moieties include Benefix, Meningitec, Refacto, Procit, Epogen, Intron A, Neupogen, Humulin, Avonex, Betaseron, Cerezyme, Genotropin, Kogenate, NeoRecormon, Gonal-F, Humalog, NovoSeven, Puregon, Norditropin, Rebif, Nutropin, Activase, Espo, Neupogen, Integrilin, Roferon, Insuman, Serostim, Prolastin, Pulmozyme, Granocyte, Creon, Hetrodin HP, Dasen, Saizen, Leukine, Infergen, Retavase, Proleukin, Regranex, Z-100, somatropin, Humatrope, Nutropin Depot, somatropin, epoetin delta, Eutropin, ranpimase, infliximab, tifacogin, oprelvekin, interferon-alpha, aldesleukin, OP-1, drotrecogin alfa, tasonermin, oprelvekin, etanercept, afelimomab, daclizumab, thymosin alpha 1, becaplermin, and A-74187. Other non-saccharide moieties which can be used include pexelizumab, anakinra, darbepoetin alfa, insulin glargine, Avonex, alemtuzumab, Leucotropin, Betaseron, aldesleukin, domase alfa, tenecteplase, oprelvekin, choriogonadotropin alfa, and nasaruplase.

Proteins and fragments thereof can be glycosylated at arginine residues, referred to as N-linked glycosylation, and at serine or threonine residues, referred to as O-linked glycosylation. In some embodiments, the protein or fragment thereof can also be modified. For example, the amino acid sequence of a protein or fragment thereof can be modified to add a site for attaching a polysaccharide moiety. The amino acid sequence of the protein or fragment thereof can be, e.g., modified to replace an amino acid which does not serve as a site for glycosylation with an amino acid which serves as a site for glycosylation. The amino acid sequence of the protein or fragment thereof can also be modified by replacing an amino acid which serves as a site for one type of glycosylation, e.g., O-linked glycosylation, with an amino acid which serves as a site for a different type of glycosylation, e.g., an N-linked glycosylation. Lastly, an amino acid residue can be added to an amino acid sequence of a protein or fragment thereof to provide a site for attaching a polysaccharide. An amino acid sequence of a protein or fragment thereof, or the nucleotide sequence encoding it, can be modified by methods known in the art.

In particularly preferred embodiments, the protein or fragment thereof is Puregon, Gamimune, Herceptin, NovoSeven, Rebif, Gonal-F, ReoPro, NeoRecormon, Genotropin, Synagis, Cerezyme, Betaseron, Humalog, Engerix-B, Remicade, Enbrel, Rituxan, Avonex, Humulin, Neupogen, Intron A, Epogen and Procit.

In one embodiment, the protein is Epogen, human EPO, and one or more of the polysaccharides associated with human EPO have been replaced by a modified polysaccharide. For example, human EPO has four glycosylation sites, three N-linked glycosylation sites at residues 24, 38 and 83 of human EPO, and an O-linked glycosylation site at residue 126. One or more of these glycosylation sites in EPO can be analyzed and replaced with a modified polysaccharide which alters an activity of EPO. In other aspects, human EPO can have a modified polysaccharide associated with it at a position which does not naturally serve as a glycosylation site in EPO. For example, one, two, three or more polysaccharides can be associated with EPO at positions not naturally associated with glycosylation in human EPO. EPO has been used to treat patient suffering from anemia, e.g., anemia associated with renal failure, chronic disease, HIV infection, blood loss or cancer. A modified polysaccharide or polysaccharides associated with EPO can be screened for various activities including increase half life, increased binding to the EPO receptor, increased stability, altered, e.g., increased, reticulocyte counts.

Methods for addition of polysaccharides or oligosaccharides to protein are known to those skilled in the art. For example, addition of sialyl Lewis acid X to antibodies for targeting purposes is described in U.S. Pat. No. 5,723,583; and modification of oligosaccharides to form vaccines is described in U.S. Pat. No. 5,370,872. A general strategy for forming protein-saccharide conjugates is outlined in U.S. Pat. No. 5,554,730.

Methods for Screening for Altered Activity

Once the modified polysaccharides have been produced, they can be rapidly screened for structure, composition, activity, or pharmacokinetics, and those polysaccharides having desirable properties selected. The effects of various polysaccharide modifications can be predicted based upon the structure of the polysaccharide and the glycomolecule. The chemical signature, e.g., structure and composition, of the modified polysaccharide can also be determined by the methods described herein and this information can be used to derive a next generation of the glycomolecule with yet another modified polysaccharide moiety.

Activities which can be screened are those properties affecting the therapeutic utility of molecules, including but not limited to altered clearance, e.g., increased or decreased clearance; altered half-life, e.g., increased or decreased half life; altered stability in vitro (shelf life) or in vivo, e.g., increased stability; altered specificity and/or efficacy (e.g., altered binding or enzymatic activity, e.g., increased or decreased binding or enzymatic activity); altered tissue distribution and targeting, e.g., increased or decreased tissue distribution or targeting; decreased toxicity; altered pK (e.g., increased pK); altered absorption rate (e.g., increased or decreased absorption rates); altered elimination rate and/or mechanism (e.g., increased or decreased elimination rates); and altered bioavailability (e.g., increased bioavailability). In addition, the following activities can be screened for: specific binding to biomolecules (for example, receptor ligands); hormonal activity; cytokine activity; inhibition of biological activity or interactions of other biomolecules (for example, agonists and antagonists of receptor binding); enzymatic activity; anti-cancer activity (anti-proliferation, cytotoxicity, antimetastasis); immunomodulation (immunosuppressive activity, immunostimulatory activity); anti-infective activity; antibiotic activity; antiviral activity; anti-parasitic; anti-fungal activity; and trophic activity.

The activity can be measured and detected using appropriate techniques and assays known in the art. Antibody reactivity and T cell activation can be considered bioactivities. Bioactivity can also be assessed in vivo where appropriate. This can be the most accurate assessment of the presence of a useful level of the bioactivity of interest. Enzymatic activity can be measured and detected using appropriate techniques and assays known in the art. Proteins and fragments thereof have been shown to influence the autophosphorylation of receptors in vitro, by assaying the amount of radiolabeled phosphate retained by the receptor before and after interaction with the protein. This can be shown using standard techniques. By influencing the phosphorylation of cell surface receptors the isolated proteins and fragments thereof can directly influence the activity of the cellular processes these receptors control. Methods to allow post translational, or peptide modification, of the proteins or fragments thereof in vitro are known. Such modifications include, but are not limited to, acylation, methylation, phosphorylation, sulfation, prenylation, further glycosylation, carboxylation, ubiquitination, amidation, oxidation, hydroxylation, adding a seleno-group to amino acid side chains (for example, selenocysteine), and fluorescent labeling.

Further in vitro analyses are used to study the effects of the glycomolecules on cell viability. For example, proteins or fragments thereof that either interrupt, stimulate, or decrease vital cellular processes may be used to infect cells, such as tumor cells, in culture. Once infected, cell growth and viability is analyzed by methods known in the art.

In vivo analyses using animal models are used to determine the effects of a glycomolecule within an intact system. For example, in the field of immunology, glycomolecules such as glycoproteins or fragments thereof can be administered to an animal and its peripheral blood monocytes are used in the generation of antibodies directed against the protein.

In the case of viral proteins for use with, for example, viral vectors, therapeutic viruses, and viral capsid delivery compositions, desired characteristics to be retained can include the ability to assemble into a viral particle or capsid and the ability to infect or enter cells. Such characteristics are useful where the delivery properties of the viral proteins are of interest, or as applied to use of the components as immunogens in vaccines.

Stability of a glycomolecule may be measured both by in vivo and in vitro techniques well known in the art. For example, blood samples may be drawn, from a host animal, at selected timepoints and antibody levels monitored and determined using ELISA kits available in the art.

In addition, other methods of screening for altered activities of a glycomolecule are well known to those skilled in the art. For example, glycoform fractions of recombinant soluble complement receptor 1 (sCR1) screened for extended half-lives in vivo are described in U.S. Pat. No. 5,456,909. In addition, antibodies having modified carbohydrate content and methods for preparation and use are described by U.S. Pat. No. 6,218,149.

### EXAMPLES

Protein Production

For each of the examples listed below, both an IgG antibody (humanized IgG4 in CHO or IgG1 in a hybridoma cell line) and erythropoietin are used as representative glycoproteins. The culturing of the cell lines is completed under sterile conditions using aseptic technique. Hybridoma or CHO cells are grown in T225 flasks from Gibco BRL in media of the following composition: 500 mls GIBCO/Invitrogen Iscove's modified media containing 10 mls 7.5% Sodium Bicarbonate, 50 mls Fetal Calf Serum (low IgG-containing), and 5 mls Glutamine/Penicillin/Streptomycin.

Cell lines (either IgG or erythropoietin producing) are split every 48-72 hours or when they appeared confluent. To complete this, the media is removed and the flask is flushed with sterile phosphate buffered saline (15 mls) to remove any media components. 2 mL of warmed Trypsin/EDTA is added to the flask to remove the adherent cells from the plastic. Once removed (˜1 min), 10 mLs of fresh media is added and the cell suspension is transferred to a conical tube and centrifuged at 1000 rpm for 5 min. The supernatant is vacuum-aspirated and fresh media is added to resuspend the cell pellet which is aliquoted into new flasks and allowed to grow. 500 mL-1 L of media containing recombinant protein is then subjected to purification as outlined below.

Protein Purification

Antibodies obtained from either CHO cells or hybridomas are purified using a protein A column (Amersham Pharmacia Biotech). Prior to column purification, the conditioned media is 0.2 μm filtered and the pH is adjusted to 7.0. The column is primed using 5 column volumes of “load” buffer (50 mM sodium phosphate, 500 mM NaCl pH 7.8), 3 column volumes of “elution” buffer (100 mM Glycine pH 3.0), and finally 5 column volumes of load buffer. Conditioned media is added to the column such that ˜10 mg of IgG is loaded per ml of resin. Then the column is washed with 5 column volumes of load buffer prior to addition of 5 column volumes of elution buffer. After elution, the protein is immediately brought to pH 7.0 using 1M Tris pH 9.0.

Human erythropoietin (EPO) is expressed as a 6×-His tagged fusion protein in an appropriate vector such as pcDNA 3.1 (Invitrogen). Conditioned media containing the His-tagged protein is 0.2 μm filtered. Prior to purification, the following buffers are run over the chelating resin. Five column volumes of “binding” buffer: 20 mM Na Phosphate, 500 mM NaCl, 5 mM Imidazole, pH 7.9, then 3 column volumes of “charge” buffer: 200 mM nickel sulfate. The column is then washed with 5 column volumes of binding buffer, the material is applied, the column washed with binding buffer, and the protein is eluted with a high imidazole buffer (20 mM Na Phosphate, 500 mM NaCl, 500 mM Imidazole, pH 7.9). Purity and amount of the proteins are assessed by silver stain gel and the micro BCA assay (BioRad).

Analysis of Glycan Structure

Glycan structures after modification are analyzed by MALDI mass spectrometry. Prior to analysis, glycan structures are typically harvested from the purified protein. Typically, 100 μg of purified glycoprotein (IgG or EPO) is digested at 37° C. for 4 hrs in a 0.1 M sodium phosphate pH 7.5 buffer containing 0.5% SDS, 1% β-mercaptoethanol, 1% NP-40 and 1000U of PNGase F (from New England Biolabs). The released glycan is purified using an activated carbon cartridge (Glyko, Inc.), eluted in 30% acetonitrile, dried and redissolved in HPLC-grade water prior to analysis.

MALDI analysis is completed on a Voyager DE STR system (Applied Biosystems) using an accelerating voltage of 22 kV. Analysis in the positive and negative modes are completed using either a 1:1 mixture of 20 mg/mL DHB in acetonitrile and a 25 mM aqueous solution of spermine or a saturated solution of 2,4,6-trihydroxyacetophenone (THAP, Fluka Chemicals) in 30% acetonitrile.

Measurement of Serum Half-Life

Increasing amounts of recombinant glycoprotein (10-500 μg) is injected i.v. via the tail vein. At time intervals ranging from 0 hr-48 hr., 100 μL of blood is withdrawn. The serum is separated via centrifugation at 1800×g for 10 minutes and analyzed using a sandwich ELISA (ZeptoMetrix, Inc.) format. Results are plotted as amount of protein vs. time after administration. Half-life is calculated using a non-compartment model.

### Example 1

### Fractionation of Glycan Isoforms

Chromatographic separation of recombinantly produced protein can be completed to isolate in a preparative manner a particular glycan isoform. Either IgG or EPO (1 mg) in 10 mM sodium phosphate pH 6.7 is added to a NucleoPac PA100 column (Dionex) at a flow rate of 1.5 mL/min and a 60 minute gradient of 0-50% of 0.3 M ammonium acetate pH 6.7 was completed. Fractions are collected, and the glycan structure is isolated and analyzed as described above. Enzymatic Modification of Recombinant Protein Ligands The following examples use ex vivo modification of glycan structures, after purification.

### Example 2

### Adding of Galactose to N-linked Sugar Structures

To 10 mg/mL of purified IgG or EPO in 50 mM Tris, 0.15M NaCl, 0.05% NaN₃ is added 100 mU/mL of β1→4 galactosyltransferase. The solution is incubated with 5 mM UDP-galactose, 10 mM MnCl₂ at 37° C. for 24-48 hrs. Incorporation is measured by taking an aliquot of the reaction mixture, isolating the glycan structure and analyzing using the MALDI procedure outlined above.

### Example 3

### Sialic Acid Capping

The glycoprotein (either modified as in example 2 or otherwise) is dissolved at 10 mg/mL in 50 mM Tris, 0.15M NaCl, 0.05% NaN₃. The solution is then incubated with 5 mM CMP-sialic acid and 100 mU/mL α2→3 (or α2→6) sialyltransferase at 32° C. for 2 days. The degree of incorporation is measured using the isolation and MALDI procedure outlined above.

### Example 4

### Addition of Other Branches

In some cases, it is desirable to increase the branching of a glycan structure, via the addition of a core α1→6 fucose or the addition of β1→4-N-acetylglucosamine. In these cases, modification is accomplished essentially the same as above. To 10 mg/mL of purified IgG or erythropoietin in 50 mM Tris, 0.15M NaCl, 0.05% NaN₃ is added 100 mU/mL of either β1→6 fucosyltransferase or β1→4-N-acetylglucosaminyltransferase III. The solution is then incubated with 5 mM of the activated sugar at 37° C. for 24-48 hrs. Incorporation is then measured by isolating the glycan structure and analyzing it using MALDI-MS.

### Example 5

### Metabolic Engineering

Synthesis of Modified Monosaccharide (ManProp):

To mannosamine hydrochloride in methanol is added 1 eq. of sodium methoxide (0.5M in methanol) and the mixture is allowed to stir for 1 hr. Then 1.1 molar equivalents of propionic anhydride is added and the mixture is allowed to stand for 3-5 hrs until the reaction is complete. The solvent is then removed via vacuum prior to peractylation.

In these cases, peracetylated monosaccharides have been shown to passively diffuse through mammalian cell membranes and undergo subsequent deacetylation by intracellular esterases, allowing efficient incorporation into proteins of modified monosaccharides. To peracetylate the monosaccharide ManProp, 100 mM acetic anhydride is added to 200 mM ManProp in pyridine, and the reaction is allowed to stir for 4 hrs. The solvent is removed and the residue is redissolved in methylene chloride, washed with water and dried. The resulting material (Ac₄ManProp) is purified using silica gel chromatography and analyzed using FAB MS and ¹H NMR.

Incorporation of Modified Monosaccharide:

To CHO cells in media is added a 100 mM ethanolic solution of the Ac₄ManProp such that the concentration of the modified monosaccharide in the media is 50-300 μM. The cells are allowed to grow to confluence and fresh monosaccharide is added with every splitting. Incorporation of the modified monosaccharide is measured after purification of the recombinant protein using MALDI-MS as described above.

To increase the level of uptake of the metabolic precursor, several parameters were varied. First, addition of cytidine, a necessary precursor of CMP-sialic acid, at concentrations of 1-10 mM, increases the level of incorporation as measured by MALDI-MS. In addition, disabling the enzyme UDP-N-acetylglucosamine 2-epimerase results in an increase in the amount of incorporation of the modified monosaccharide. Synthetic monosaccharides likely compete with the physiological precursor N-acetylmannosamine and its metabolic products for the sialic acid machinery, resulting in only moderate expression of modified sailic acid derivatives on the surface of recombinant glycoproteins. Thus, a cell lacking this enzyme can only generate sialic acid moieties through a scavenge pathway, i.e. modified monosaccharides added to the media, resulting in a larger degree of incorporation. This enzyme can be disabled by methods commonly known in the art.

Finally, incorporation of modified sialic acid monosaccharide analogues can be increased via the addition of a glycosyltransferase, such as β1→4 galactosyltransferase or α2→3 (or α2→6) sialyltransferase. To transfect CHO cells producing recombinant protein, the Lipofectamine 2000 protocol from Invitrogen is followed. In this case, CHO cells are seeded at 0.5×10⁵ cells per well in a 24 well plate one day before transformations are carried out so that the cells would be roughly 90% confluent on the day of transformation. Transformations are done in triplicate for 2 clones containing the erythropoietin gene in a PCDNA3.1 vector from Invitrogen. Fifty μl of F10 media is mixed with 0.8-1 μg of DNA. In a separate tube, 50 μl of F10 media is mixed with 2-3 μl of lipofectamine. Mixtures are incubated at room temperature for 5 minutes, mixed together, and incubated for an additional 20 minutes at room temperature. Each DNA-lipofectamine mixture is then added to one well of the 24 well plate. After 4 hours of incubation at 37° C., the media is removed from the wells and replaced with fresh media. Twenty-four hours after transformation, the media is replaced with selective media containing 500 μg/ml geneticin (purchased from Invitrogen). Cells are grown for several days, and media was harvested to assay for erythropoietin using an ELISA kit. Protein expression of the relevant transferase is confirmed, and cell populations are expanded and clonal populations were established.

### Example 6

### Glyco-Modification of an Anti-MHC Antibody

A hybridoma cell line expressing an anti-MHC antibody (OKT3) was grown in roller bottles in Iscove's modified Dulbecco's medium containing 10% Ultra-low IgG fetal bovine serum (Gibco). 1,3,4,6,-tetra-O-acetyl-N acylmannosamine, or derivative thereof, to a final concentration of 10-50 μM. The cells were allowed to grow with fluid renewal every 2-3 days and at these time points, antibody was harvested from the spent media.

Media containing the antibody was run over a protein A column (Sepharose CL4B fast flow) to purify the antibody. Bound antibody was washed with ice cold PBS and 10 mM Tris pH 8.0. following the washing steps, the antibody was eluted with 100 mM glycine pH3 and immediately brought to pH 7.0 with 1M Tris. Antibody purity and concentration were assessed by denaturing a portion of the preparation and running a silver stain gel as well as determining the A₂₈₀ (OD₂₈₀ of 1=0.75 mg/ml).

To assess the glycosylation pattern of the OKT antibody, 100 μg of the preparation was denatured and digested with PNGase F overnight at 37° C. After digestion, the glycan was purified via an activated charcoal column. Glycoforms were assessed by capillary electrophoresis using a 50 mM phosphate pH 2.5 running buffer and/or via MALDI mass spectrometry using a aqueous saturated solution of DHB matrix containing 300 mM spermine HCL.

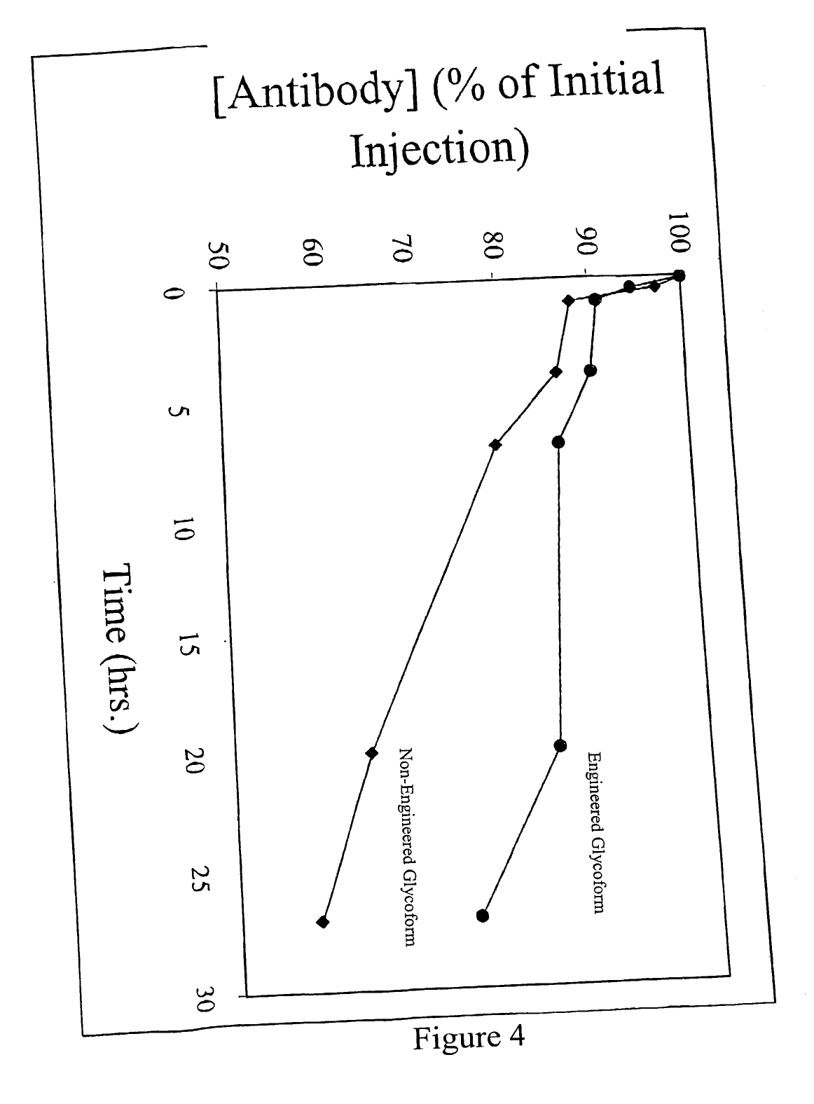

In order to assess the in vivo half-life of the glyco-modified antibody, 100 μg/kg purified antibody was injected intravencously into New Zealand rabbits. Blood samples were drawn at selected timepoints from 0-100 hours post-injection. Antibody levels were determined using an IgG-specific ELISA kit (FIG. 4).

The references, patents and patent applications cited herein are incorpoated by reference. Modifications and variations of these methods and products thereof will be obvious to those skilled in the art from the foregoing detailed description and are intended to be encompassed within the scope of the appended claims.

We claim:

1. A method for producing a molecule, comprising a first, non-saccharide moiety, and a second, polysaccharide, moiety, the method comprising:
determining the chemical composition and structure of all or a portion of the second moiety by a method which includes comparison of one or more properties of the second moiety with a database,
modifying the structure of the second moiety to provide a modified second moiety, and
evaluating or screening the molecule having the modified second moiety for a biological activity or other chemical or physical property.

2. The method of claim 1, wherein the second moiety has an activity defined by comparison to a database of known polysaccharides, and the evaluation or screening includes evaluating the molecule for unaltered or altered, enhanced or optimized biological activity of the modified second moiety.

3. The method of claim 1, wherein the modification of the second moiety includes using the determined composition and structure of the second moiety to produce the modified second moiety or a portion thereof using enzymatic, chemical, or chemoenzymatic synthesis.

4. The method of claim 1, wherein the modification includes changing one or more of the identity, number, or linkage of one or more chemical units in the second moiety.

5. The method of claim 1, wherein the modification includes changing the number of branches in the second moiety.

6. The method of claim 5 wherein the second moiety or portion thereof is modified by increasing the branching of the second moiety.

7. The method of claim 5, wherein the second moiety of portion thereof is modified by decreasing the branching of the second moiety.

8. The method of claim 1, wherein the modification includes enzymatic cleavage or enzymatic addition of one or more chemical units.

9. The method of claim 1, wherein the modification is effected by altering a synthetic process which produces a polysaccharide moiety by adding an excess of a substrate or intermediate in a synthetic reaction.

10. The method of claim 8, wherein the second moiety or portion thereof is modified by enzymatic cleavage using a degrading enzyme selected from an α-galactosidase which cleaves a α1→3 glycosidic linkage after a galactose, a β-galactosidase which cleaves a β1→4 linkage after a galactose, an α2→3 sialidase which cleaves a α2→3 glycosidic linkage after a sialic acid, an α2→6 sialidase which cleaves after an α2→6 linkage after a sialic acid, an α1→2 fucosidase which cleaves a α1→2 glycosidic linkage after a fucose, a α1→3 fucosidase which cleaves a α1→3 glycosidic linkage after a fucose, an α1→4 fucosidase which cleaves a α1→4 glycosidic linkage after a fucose, an α1→6 fucosidase which cleaves an α1→6 glycosidic linkage after a fucose, a N-acetylglucosiaminidase which cleaves a β1→2 glycosidic linkage after a GlcNAc, a N-acetylglucosiaminidase which cleaves a β1→4 glycosidic linkage after a GlcNAc, and a N-acetylglucosiaminidase which cleaves a β1→6 linkage after a GlcNAc.

11. The method of claim 8, wherein the second moiety or portion thereof is modified by enzymatic addition of one or more monosaccharides using an enzyme selected from the group consisting of a N-acetylglucosaminyltransferase, a galactosyltransferase, a sialyltransferase and a fusosyltransferase.

12. The method of claim 11, wherein the enzyme is β1→4 N-acetylglucosaminyltransferase in the presence of β1→4 N-acetylglucosamine.

13. The method of claim 11, wherein the enzyme is β1→4 galactosyltransferase in the presence of β1→4 galactose.

14. The method of claim 11, wherein the enzyme is α2→3 sialyltransferase in the presence of α2→3 sialic acid.

15. The method of claim 11, wherein the enzyme is α2→6 sialyltransferase in the presence of α2→6 sialic acid.

16. The method of claim 11, wherein the enzyme is αl→6 fucosyltransferase in the presence of α1→6 fucose.

17. The method of claim 11, wherein the enzyme is α1→3 fucosyltransferase in the presence of α1→3 fucose.

18. The method of claim 1, wherein a first moiety is analyzed for an existing polysaccharide moiety and the modification is performed on the existing polysaccharide moiety on the first moiety.

19. The method of claim 1, wherein a polysaccharide moiety not naturally attached to the first moiety is selected and formed as a modified second moiety on the first moiety.

20. The method of claim 1, wherein the first moiety includes an existing polysaccharide naturally attached to it which has been removed, and a polysaccharide not naturally attached to the first moiety is added as a modified second moiety.

21. The method of claim 20, wherein the modified second moiety is added at a position in the first moiety where the naturally existing polysaccharide had previously been attached or at a position in the first moiety where no naturally existing polysaccharide had previously been attached.

22. The method of claim 1, wherein the first moiety is not naturally associated with a polysaccharide.

23. The method of claim 1, wherein the molecule is selected from the group consisting of a glycoprotein, a proteoglycan, a glycopolypeptide, glycopeptide, glycoamino acid, and a glycolipid.

24. The method of claim 1, wherein the activity of the molecule is unaltered, increased, decreased, eliminated by the modified second moiety.

25. The methods of claim 24, wherein the activity of the molecule is increased by the modified second moiety.

26. The method of claim 25, wherein the activity which is increased is selected from the group consisting of half-life, stability, IC₅₀ (ED₅₀), specificity, efficacy, absorption time, elimination time, tissue targeting, bioavailability and binding.

27. The method of claim 24, wherein the activity of the molecule is decreased or eliminated by the modified second moiety.

28. The method of claim 27, wherein the activity which is decreased or eliminated is a side effect associated with therapy, toxicity, immunogenicity, clearance and organ accumulation.

29. The method of claim 1, wherein the first moiety is a protein or fragment thereof.

30. The method of claim 29, wherein the modified second moiety is an N-linked polysaccharide.

31. The method of claim 30, wherein the N-linked polysaccharide is selected from the group consisting of simple, complex, hybrid and high mannose polysaccharides.

32. The method of claim 29, wherein the modified second moiety is an O-linked polysaccharide.

33. The method of claim 1, wherein the first moiety and the modified second moiety are attached by chemical, enzymatic or chemoenzymatic ligation.

34. The method of claim 29, wherein the polypeptide or fragment thereof is modified by modifying the amino acid sequence to add a site for attaching the second moiety.

35. The method of claim 34, wherein the protein or fragment thereof is modified to replace an amino acid which does not serve as a site for attaching a polysaccharide or serves as a site for attaching a one type of polysaccharide with another amino acid which serves as a site for attaching a different type of polysaccharide.

36. The method of claim 34, wherein the protein or fragment thereof is modified by adding to the amino acid sequence an additional amino acid which serves as a site for attaching a polysaccharide.

37. A molecule produced by the method of claim 1.

38. A molecule of claim 37, wherein the modified second moiety is part of a larger polysaccharide.

39. A method for producing a molecule, comprising a first, non-saccharide moiety, and a second, polysaccharide, moiety, the method comprising:
determining the chemical composition and structure of all or a portion of the second moiety by a method which includes comparison of one or more properties of the second moiety with a database,
modifying the structure of the second moiety to provide a modified second moiety,
evaluating or screening the molecule having the modified second moiety for a biological activity or other chemical or physical property, and
attaching the modified second moiety to a different first moiety.

40. The method of claim 39, wherein the second moiety has an activity defined by comparison to a database of known polysaccharides, and the evaluation or screening includes evaluating the molecule for altered, enhanced or optimized biological activity of the modified second moiety.

41. The method of claim 39, wherein the modification of the second moiety includes using the determined composition and structure of the second moiety to produce the modified second moiety or a portion thereof using enzymatic, chemical, or chemoenzymatic synthesis.

42. The method of claim 39, wherein the modification includes changing one or more of the identity, number, or linkage of one or more chemical units in the second moiety.

43. The method of claim 39, wherein the modification includes changing the number of branches in the second moiety.

44. The method of claim 43, wherein the second moiety or portion thereof is modified by increasing the branching of the second moiety.

45. The method of claim 43, wherein the second moiety of portion thereof is modified by decreasing the branching of the second moiety.

46. The method of claim 39, wherein the modification includes enzymatic cleavage or enzymatic addition of one or more chemical units.

47. The method of claim 39, wherein the modification is effected by altering a synthetic process which produces a polysaccharide moiety by adding an excess of a substrate or intermediate in a synthetic reaction.

48. The method of claim 46, wherein the second moiety or portion thereof is modified by enzymatic cleavage using a degrading enzyme selected from an α-galactosidase which cleaves a α1→3 glycosidic linkage after a galactose, a β-galactosidase which cleaves a β1→4 linkage after a galactose, an α2→3 sialidase which cleaves a α2→3 glycosidic linkage after a sialic acid, an α2→6 sialidase which cleaves after an α2→6 linkage after a sialic acid, an α1→2 fucosidase which cleaves a α1→2 glycosidic linkage after a fucose, a α1→3 fucosidase which cleaves a α1→3 glycosidic linkage after a fucose, an α1→4 fucosidase which cleaves a α1→4 glycosidic linkage after a fucose, an α1→6 fucosidase which cleaves an α1→6 glycosidic linkage after a fucose, a N-acetylglucosiaminidase which cleaves a β1→2 glycosidic linkage after a GlcNAc, a N-acetylglucosiaminidase which cleaves a β1→4 glycosidic linkage after a GlcNAc, and a N-acetylglucosiaminidase which cleaves a β1→6 linkage after a GlcNAc.

49. The method of claim 46, wherein the second moiety or portion thereof is modified by enzymatic addition of one or more monosaccharides using an enzyme selected from the group consisting of a N-acetylglucosaminyltransferase, a galactosyltransferase, a sialyltransferase and a fusosyltransferase.

50. The method of claim 49, wherein the enzyme is β1→4 N-acetylglucosaminyltransferase in the presence of β1→4 N-acetylglucosamine.

51. The method of claim 49, wherein the enzyme is β1→4 galactosyltransferase in the presence of β1→4 galactose.

52. The method of claim 49, wherein the enzyme is α2→3 sialyltransferase in the presence of α2→3 sialic acid.

53. The method of claim 49, wherein the enzyme is α2→6 sialyltransferase in the presence of α2→6 sialic acid.

54. The method of claim 49, wherein the enzyme is α1→6 fucosyltransferase in the presence of α1→6 fucose.

55. The method of claim 49, wherein the enzyme is α1→3 fucosyltransferase in the presence of α1→3 fucose.

56. The method of claim 39, wherein the first moiety includes an existing polysaccharide naturally attached to it which has been removed, and a polysaccharide not naturally attached to the first moiety is added as a modified second moiety.

57. The method of claim 56, wherein the modified second moiety is added at a position in the first moiety where the naturally existing polysaccharide had previously been attached or at a position in the first moiety where no naturally existing polysaccharide had previously been attached.

58. The method of claim 39, wherein the first moiety is not naturally associated with a polysaccharide.

59. The method of claim 39, wherein the molecule is selected from the group consisting of a glycoprotein, a proteoglycan, a glycopolypeptide, glycopeptide, glycoamino acid, and a glycolipid.

60. The method of claim 39, wherein the activity of the molecule is increased, decreased, eliminated by the modified second moiety.

61. The methods of claim 60, wherein the activity of the molecule is increased by the modified second moiety.

62. The method of claim 61, wherein the activity which is increased is selected from the group consisting of half-life, stability, IC₅₀ (ED₅₀), specificity, efficacy, absorption time, elimination time, tissue targeting, bioavailability and binding.

63. The method of claim 60, wherein the activity of the molecule is decreased or eliminated by the modified second moiety.

64. The method of claim 63, wherein the activity which is decreased or eliminated is a side effect associated with therapy.

65. A method of producing a first molecule comprising a first non-saccharide moiety and a second polysaccharide moiety, the method comprising:
selecting a modified second moiety which has been modified based upon its ability to confer a desired property on a second molecule, wherein the modified second moiety has been modified based upon its chemical structure,
providing the modified second moiety which has been modified based upon its chemical structure and composition; and
producing a first molecule which comprises a first non-saccharide moiety and the modified second moiety, wherein the modified second moiety alters an activity of the first moiety, thereby producing a first molecule.
